(19) United States
(12) Patent Application Publication
(10) Pub. No.: US 2023/0295679 A1
(43) Pub. Date: Sep. 21, 2023

(54) PROCESS FOR PREPARING ALKYL POLYGLUCOSIDES, AND ALKYL POLYGLUCOSIDES OBTAINED ACCORDING TO THE PROCESS

(71) Applicants: INSTITUT NATIONAL DES SCIENCES APPLIQUEES DE TOULOUSE, Toulouse (FR); Institut National de Recherche Pour l'Agriculture, l'Alimentation et l'Environnement, Paris (FR); Centre National de la Recherche Scientifique (CNRS), Paris (FR)

(72) Inventors: Etienne SEVERAC, Toulouse (FR); Claire MOULIS, Toulouse (FR); David GUIEYSSE, Toulouse (FR); Sandrine MOREL, Toulouse (FR); Magali REMAUD-SIMEON, Toulouse (FR)

(73) Assignees: INSTITUT NATIONAL DES SCIENCES APPLIQUEES DE TOULOUSE, Toulouse (FR); Institut National de Recherche Pour l'Agriculture, l'Alimentation et l'Environnement, Paris (FR); Centre National de la Recherche Scientifique (CNRS), Paris (FR)

(21) Appl. No.: 18/013,713

(22) PCT Filed: Jul. 2, 2021
(86) PCT No.: PCT/FR2021/051220

§ 371 Date: Dec. 29, 2022

(30) Foreign Application Priority Data
Jul. 2, 2020 (FR) FR2007021

(51) Int. Cl. C12P 19/44; C12N 9/10

(52) U.S. Cl. CPC C12P 19/44; C12N 9/1051; C12Y 204/01005

(57) ABSTRACT

The present invention relates to a process for preparing an alkyl polyglucoside by enzymatic catalysis, using sucrose or an analogue thereof as substrate and making it possible to obtain a large diversity of alkyl polyglucosides in terms of size and structure of the glucoside part thereof, making possible the obtaining of an alkyl polyglucoside with a number of glucosyl units that can be adjusted from 2 to 200 glucosyl units. The process also makes it possible to adjust the linear or branched structure of the carbohydrate part of the alkyl polyglucoside obtained, and also the nature of the glycosidic bonds linking the glucose residues within the carbohydrate part.

### FIELD OF THE INVENTION

The present invention relates to the field of enzymatic preparation of alkyl polyglucosides. The present invention also relates to the alkyl polyglucosides that can be obtained by said method.

### TECHNICAL BACKGROUND

Surfactants have long been used in a large number of fields such as pharmaceuticals, cosmetics, hygiene and care products or industrial formulations due to their varied properties: foaming, emulsifying, dispersing, detergent, etc.

Surfactants consist structurally of a hydrophobic part and a hydrophilic part, the latter being positively or negatively charged, or even uncharged.

Alkyl polyglucosides (APGs) are glucolipidic surfactants, the polar head of which is formed of glucosyl residues and a more or less long carbon chain, typically having 1 to 18 carbon atoms.

APGs have interesting surface properties, biodegradability and harmlessness to the skin and mucous membranes, which gives them a certain interest for many industrial applications.

APGs are conventionally synthesised via chemical pathways. The most common procedures for synthesis of APGs include the Fischer method or Koenig-Knorr method, or the methods of Schmidt.

The Fischer method is the simplest to implement and is conventionally used in industry when the selectivity of the synthesis is not a sought criterion. Depending on the nature of the carbohydrate and the fatty alcohol, the Fischer reaction can be carried out in one or two steps. The carbohydrate is dissolved in an excess of alcohol in the presence of an acid catalyst and at high temperature.

The obtaining of APGs for which the carbon chain is large requires a step in which a small alkyl typically a butyl-, is exchanged with an alcohol for which the carbon chain is larger, thus enabling the problems of reactant solubility to be overcome.

Fischer synthesis leads to the obtaining of a mixture which can be extremely complex, of alkyl mono-, di-, tri- and oligoglucosides.

The glucosyl units constituting the APG can be present in the form of α or β anomers, and pyranoside and furanoside isomers.

For reasons of reaction kinetics and reaction product incompatibilities (low solubility of carbohydrates in fatty alcohols), the average degree of polymerisation of the glucoside part of commercial APGs is still currently less than 2 (Ulvenlund et al., 2016), and is typically between 1.3 and 1.6.

However, for certain applications, APGs for which the glucoside part is particularly long are required and requested by industrial stakeholders.

In view of the above, the development of pathways for efficient synthesis, enabling access to APGs for which the glucoside part is particularly long, is a major challenge.

In recent years, notable efforts have been dedicated to the development of enzymatic pathways for synthesis of APGs.

The most-studied enzymes in this respect are enzymes belonging to the family of glycoside hydrolases and more particularly those of the family of p-glycosidases. The latter are enzymes without co-factors which naturally hydrolyse osidic bonds of the β type present in the polysaccharides in order to produce mono- or oligosaccharides. In vitro, these enzymes are capable of using a glycosyl donor and catalysing the transfer of glycosyl onto the free hydroxyl of an acceptor molecule.

The synthesis of alkyl-polyglucosides catalysed by p-glycosidases, in general maintains a β bond configuration between the sugars and the alkyl part (these are referred to as β-alkyl-polyglucosides). This synthesis is possible either by hydrolysis reversal (where the glucosyl donor can be cellulose or β-glucan), or by transglycosylation (where the glycosyl donors are natural polysaccharides or activated carbohydrates such as methyl-β-D-glucopyranoside, pNP-β-D-glucopyranoside or other aryl-glucosides).

Other families of enzymes have made it possible to obtain alkyl-polyglucosides.

Bousquet et al. have demonstrated the possibility of obtaining α-alkyl-polyglucosides by using an α-transglucosylase, from maltodextrins on butanol (Bousquet et al., 1998; Bousquet et al., 1999).

Dahiya and co-workers have succeeded in producing 1-O-hexyl-α-D-mono, di- and tri-α-glucopyranosides from hexanol or octanol and sucrose by using a strain of Microbacterium paraoxydans having a membrane transglucosylation activity of the amylo-sucrase type (Dahiya et al., 2015).

Ochs and co-workers have produced pentyl- and octyl-polyxylosides (alkyl-polyxylosides) by transglycosylation reaction between pentanol, octanol and xylanes catalysed by various xylanases (Ochs et al., 2011; Remond et al., 2012).

In each case, the difference in the chemical nature of the reactants (carbohydrates and fatty alcohols) as well as the very hydrophilic nature of the sugars and, by contrast, very hydrophobic nature of fatty acids, does not allow sufficient concentrations to be obtained of the two substrates in the same phase, which broadly limits the production yields and the sizes of glycosidic heads to several units.

An alternative pathway is to use commercial APGs having a glucoside part of moderate size (typically with a degree of polymerisation less than 3) and to lengthen the glucoside part via an enzymatic pathway.

In this respect, cyclodextrin glucanotransferases (CGTases) have broadly been described for their capacity to produce APGs with low DP by transglucosylation reaction.

The CGTases belong to the GH13 and GH57 families of glycoside hydrolases, and are specific to the formation of α-1,4 glucosidic bonds. These enzymes catalyse four types of reaction, from starch or amylose: cyclisation, coupling, disproportionation and hydrolysis. In the case where a non-glucoside molecule carrying one or more glucosyl residues is introduced into the medium, the CGTases are likely to orient their activity towards a coupling and/or disproportionation on this acceptor.

Okada and co-workers were the first to use the coupling capabilities of such enzymes in order to elongate glycolipids (sucrose esters). They obtained the addition of 1 to 3 glucosyl units on sucrose laurate (Okada et al., 2007).

The capability of CGTases to extend butanol, octanol and dodecanol maltosides has been demonstrated by Zhao and co-workers in 2008 (Zhao et al., 2008).

The APGs used could only be extended by 2 to 3 glucosyl units by CGTase of Bacillus stearothermophilus starting from dextrins with 10 to 15 glucosyl units.

Svensson and co-workers compared the coupling and disproportionation activities of CGTases from Bacillus macerans and from Thermoanaerobacter (Toruzyme 3.0L, Novozymes A/S, Denmark) on dodecyl-maltoside (C12G2, in other words an APG composed of a C12 alkyl and a glucoside part comprising two glucosyl units) from α-cyclodextrins (Svensson et al., 2009). It has been possible to show that the differences between coupling/disproportionation reaction properties of the two CGTases, enabled the reaction products to be modulated. Hence, mixtures of products carrying 1 to 20 glucosyl units have been obtained with profiles varying according to the CGTases (C12G8 and C12G14 for CGTase of B. macerans, a slightly disproportionate enzyme and a +/−homogeneous distribution of C12G1 to C12G20 for the CGTase of Thermoanaerobacter, a very disproportionate enzyme).

The obtaining of various products is controlled by the reaction kinetics (Svensson et al., 2011), the coupling reactions being quicker than those for disproportionation. Hence, it has been possible to control the profile of products in extended APGs by controlling the flow through a fixed bed reactor containing CGTase of B. macerans immobilised on Eupergit C: a rapid flow promotes the coupling product (C12G8) whereas a slow flow does not favour C12G8 and favours the double or triple coupling products and the disproportionation products (C12G14, C12G20).

Paul and co-workers have used a glucanotransferase of the GH57 family, to extend dodecanol maltoside from starch. APGs carriers with 24 to 26 glucosyl units (C12G24 to C12G26) were identified by SM analysis (Paul, et al., 2015).

Glucansucrases (GS) from the GH13 and GH70 families can also be used for such glucosylation reactions.

Starting from sucrose, glucansucrases catalyse the synthesis of glucans generally having very high molar masses and varied structures due to the presence of different types of osidic bond (α-1,2, α-1,3, α-1,4 and/or α-1,6) as well as their location in the polymer. Isomers of sucrose and of glucose are also products from sucrose, but generally in much lower quantities than the polymer.

Several glucansucrases of the families GH13 and GH70 have been used in acceptor reactions with the aim of extending the glucoside part of alkyl monoglucoside by a short alkyl group (1 to 8 carbon atoms) from sucrose.

Hence, the extracellular dextran-sucrases of Leuconostoc mesenteroides NRRL-B512F have, since 1966, allowed the transfer of 1 to 4 glucosyl units to the 1-O-methyl-α-D-glucoside.

Alternansucrase from L. mesenteroides NRRL B-1355 catalysed the transfer of a single glucosyl unit onto 1-O-methyl-α- and p-D-glucosides (Côté et al., 1982).

More recently, Richard and co-workers studied the effect of the length of the alkyl chain on the capacity of dextransucrase to glucosylate the alkyl-monoglucosides and have shown that, if glucansucrases of L. mesenteroides B-512F, B-1299, B-1355 and B-23192 are capable of glucolysating 1-O-butyl-α-glucoside (by adding 1 to 3 additional glucosyl units according to the α-1,6 bonds), only the alternansucrases of L. mesenteroides B-1355 and B-23192 have extended 1-O-octyl-α-glucoside (Richard et al., 2003).

The known methods are therefore limited, in particular since they do not make it possible to easily modulate both the number and nature of the bonds connecting the glucosyl units within the alkyl polyglucosides obtained.

Furthermore, the majority of known methods cannot have an alkyl glucoside as substrate having long alkyl chains, typically greater than 8 carbon atoms.

There is therefore a need for a method for preparing alkyl glucosides enabling access to a large range of molecular architectures by using a reusable and inexpensive substrate such as sucrose.

In particular, there is also a need for a method that makes it possible to modulate both the number of glucosyl units within the glucoside part, but also the nature of the osidic bonds connecting said glucosyl units, while making it possible to obtain alkyl polyglucosides with the long glucoside chains, and this even if the alkyl glucoside used as substrate has a large-sized alkyl group.

### BRIEF DESCRIPTION OF THE FIGURES

FIG. 1 shows the reaction diagram for enzymatic elongation of the glucosidic part of an alkyl monoglucoside using α-transglucosylases of the GH70 family that are active on sucrose.

FIGS. 2A and 2B show the elongation profiles for the substrate C8G1 obtained with α-transglucosylases of the GH70 family which use sucrose as glucosyl donor. FIG. 2A FIG. 2A shows the profiles obtained with glucansucrases from [C8G1]=30 mM and [sucrose]=585 mM for glucansucrases DSR-M Δ1 (SEQ ID NO: 1), DSR-M Δ5 (SEQ ID NO: 2), DSR-M Δ5 W624A (SEQ ID NO: 3), GS-B (SEQ ID NO: 5), GS-C(SEQ ID NO: 6), GS-D (SEQ ID NO: 9) and GS-FS Δ1 (SEQ ID NO: 10) and 1170 mM sucrose for the enzymes GS-A SEQ ID NO:4, GS-D (SEQ ID NO:7), DSR-G (SEQ ID NO:11), DSR-G CD1 (SEQ ID NO:12). The glucansucrases are used in reaction at 1 U·mL⁻¹. FIG. 2B shows the profiles obtained with branching enzymes BRS-A (SEQ ID NO: 17), BRS-B Δ1 (SEQ ID NO: 18), BRS-C (SEQ ID NO: 19), BRS-D Δ1 (SEQ ID NO: 20), BRS-E Δ1 (SEQ ID NO: 21), BRS-F (SEQ ID NO: 22), DSR-G CD2 (SEQ ID NO: 23), GBD-CD2 (SEQ ID NO: 16), from [C8G1]=20 mM and [sucrose]=585 mM. The branching enzymes are used in reaction at 1 U·mL⁻¹

FIG. 3 compares the profiles of the elongation reaction of C8G1 catalysed by GS-C(SEQ ID NO: 6) in the presence of 200 g·L⁻¹ sucrose and 200 g·L⁻¹ α-cyclodextrins and cyclodextrin glucanotransferases (CGTase) of Bacillus macerans (an enzyme not according to the invention) in the presence of 200 g·L⁻¹ α-cyclodextrins (substrate not according to the invention).

FIG. 4 compares the elongation reaction profiles of C8G1 catalysed by glucansucrase DSR-M Δ1 alone (SEQ ID NO: 1) and catalysed by glucansucrase DSR-M Δ1 and branching enzymes BRS-A (SEQ ID NO:17) or BRS-B Δ1 (SEQ ID NO:18) used sequentially.

FIG. 5 FIG. 5 shows the elongation profile for alkyl glucosides of increasing size, obtained with glucansucrase DSR-M Δ1 (SEQ ID NO: 1) with initial sucrose concentrations of 200 g·L⁻¹. BoxA: [C8G1]=30 mM; Box B: [C10G1]=10 mM; Box C: Triton CG110 (Dow Chemicals, USA) at 15 g·L⁻¹; Box D: [C12G1]=5 mM; Box E: [C12G2]=10 mM, Box F: [C16G2]=5 mM.

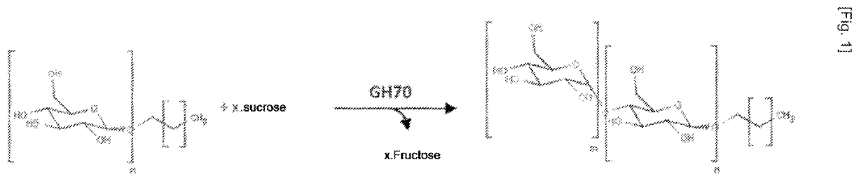

### DETAILED DESCRIPTION OF THE INVENTION

The aim of the present invention is to overcome the disadvantages of the prior art and to provide a method for preparing an alkyl polyglucoside by enzymatic catalysis, said method being economical because it uses sucrose or one of its analogues as a substrate and making it possible to obtain a wide-range of alkyl polyglucosides in terms of size and structure of their glucosidic part.

Hence, the method of the invention makes it possible to obtain an alkyl polyglucoside with a number of glucosyl units that can be modulated from 2 to 200 glucosyl units.

The method also makes it possible to modulate the linear or branched structure of the glucoside part of the alkyl polyglucoside obtained, as well as the nature of the alpha-1,2; alpha-1,3; alpha-1,4 or alpha-1,6 osidic bonds connecting the glucosyl residues within the glucoside part.

Significantly, the method of the invention also makes it possible to prepare alkyl polyglucosides having a large alkyl group, for example greater than 8 carbon atoms.

This diversity is very advantageous for producing novel alkyl polyglucosides of interest in the detergent and cosmetic industries.

Another aim of the invention is to provide alkyl polyglucosides with a wide diversity in terms of size and structure of the glucosidic part, and in particular alkyl polyglucosides for which the size of the alkyl group is large, for example greater than 8 carbon atoms.

These aims are achieved by the invention which will be described below.

The first object of the invention is a method for preparing an alkyl polyglucoside of formula (I)

[Glc]ₘ-[Glc]ₙ(-O—R) (I)

wherein:
R represents a linear or branched, saturated or unsaturated alkyl group, comprising between 8 and 20 carbon atoms,
[Glc]ₘ-[Glc]ₙ represents a linear or branched glucoside part comprising n+m glucosyl units, n+m being between 3 and 200,
said method comprising at least a step i) of elongating the glucoside chain of an alkyl glucoside of formula (II)

[Glc]ₙ(-O—R) (II)

in which:
R is as defined in formula (I),
[Glc], represents a glucoside part comprising n glucosyl units,
n being between 1 and 15.
said step comprising placing said alkyl glucoside of formula (II) in contact with at least one α-transglucosylase of the GH70 family in the presence of sucrose or a sucrose analogue.

Through the method according to the invention, it is now possible to prepare a wide range of alkyl polyglucosides.

Significantly, the method makes it possible to control the size, the branched or unbranched structure and the nature of the osidic bonds within the glucoside part of the alkyl polyglucoside obtained, and this even when the alkyl group of the alkyl glucoside used as substrate is large.

The α-transglucosylases of the GH70 family are water-soluble enzymes naturally catalysing hydrophilic substances, and for which the activity is a priori strongly limited in the presence of fatty material.

The inventors have discovered that, surprisingly, α-transglucosylases of the GH70 family are capable of accepting, as substrate, a C8-C20 alkyl glucosyl, in other words a poorly soluble compound comprising a hydrophobic part which is unfavourable to the action of this type of enzyme, and efficiently catalysing the elongation of its glucoside part.

Glucoside part According to the invention, the expression “glucoside part”, designates a linear or branched polymer formed from glucosyl units bonded together by osidic bonds.

According to the invention, the terms “glycosidic bond” or “glucosidic bond” or “osidic bond” can be used interchangeably and designate the covalent bond that bonds one glucosyl to another adjacent glucosyl.

The glucoside part [Glc]ₘ-[Glc]ₙ of the alkyl polyglucoside of formula (I) comprises two glucoside fractions, [Glc]ₘ and [Glc]ₙ.

The glucoside fraction [Glc], corresponds to the n glucosyl units of the glucoside part of the alkyl glucoside of formula (II) used as substrate in the method of the invention. The glucoside fraction [Glc]m corresponds to the m glucosyl units added on the glucoside part [Glc], of the alkyl glucoside of formula (II) during the glucoside elongation step of the method of the invention.

n+m, in other words the number of glucosyl units in the glucoside part [Glc]ₘ-[Glc]ₙ of the alkyl polyglucoside of formula (I) is advantageously between 3 and 150, preferably between 3 and 100, more preferably between 3 and 30, more preferably between 3 and 25. Preferably, n+m is between 5 and 50, and can be, in particular, between 5 and 40, more particularly between 5 and 30, in particular between 5 and 25. Advantageously, n+m is between 7 and 200, preferably between 8 and 200, preferably between 9 and 200, preferably between 10 and 200.

m, in other words the number of glucosyl units in the glucoside fraction [Glc]ₘ of the glucoside part [Glc]ₘ-[Glc]ₙ of the alkyl polyglucoside of formula (I), or in other words the number of glucosyl units added on the glucoside part [Glc], of the alkyl glucoside of formula (II) during the implementation of the method of the invention, is advantageously greater than 3. Preferably, m is between 3 and 150, preferably between 3 and 100, more preferably between 3 and 30.

n, in other words the number of glucosyl units in the glucoside fraction [Glc], of the glucoside part [Glc]ₘ-[Glc]ₙ of the alkyl polyglucoside of formula (I), or in other words the number of glucosyl units of the glucoside part [Glc], of the alkyl glucoside of formula (II), is advantageously between 1 and 8, preferably between 1 and 5, more preferably between 1 and 3. Preferably, n is equal to 1 or 2.

Types of Bond

According to the invention, the expression “a bond” designates a covalent bond which bonds carbon atom 1 of a glucosyl unit in its a configuration; and the expression “p bond” designates a covalent bond which bonds carbon atom 1 of a glucosyl unit in its p configuration.

Within the glucoside fraction [Glc], of the alkyl polyglucoside of formula (I) or the glucoside part [Glc], alkyl glucoside of formula (II), the glucosyl unit adjacent to the alkyl group is bonded to the alkyl group by an α bond or a β bond, preferably by a β bond.

In other words, in the alkyl polyglucoside of formula (I) and the alkyl glucoside of formula (II), the glucosyl unit adjacent to the alkyl group is in α or β configuration, preferably in p configuration.

The other glucosyl units of the glucoside fraction [Glc], of the alkyl polyglucoside of formula (I) or of the glucoside part [Glc], alkyl glucoside of formula (II) are bonded together by α and/or β glucosidic bonds, preferably by a glucosidic bonds.

The m adjacent glucosyl units within [Glc]ₘ are bonded to one another by a glucosidic bonds.

In the present document, the term “α-1,3 bond” designates the covalent bond which bonds carbon atom 1 of a glucosyl unit in its a configuration and carbon 3 of another adjacent glucosyl unit. The term “α-1,2 bond” designates the covalent bond which bonds carbon atom 1 of a glucosyl unit in its a configuration and carbon 2 of another adjacent glucosyl unit. The term “alpha-1,6 bond” designates the covalent bond which bonds carbon atom 1 of a glucosyl unit in its alpha configuration and carbon 6 of another adjacent glucosyl unit.

The alpha-glucosyl units are preferably alpha-D-glucosyl units.

The analysis of the glycosidic bonds of an alkyl polyglucoside can be carried out using any method known to a person skilled in the art.

For example, the glucosidic bonds can be analysed by nuclear magnetic resonance (NMR).

Step i) can be carried out with an alkyl glucoside of formula (II), essentially formed of alkyl monoglucoside molecules, alkyl diglucoside molecules, or a mixture thereof, said mixture advantageously having an average degree of polymerisation between 1 and 2, preferably between 1.3 and 1.6.

The mixture is essentially composed of alkyl monoglucoside molecules and alkyl diglucoside molecules, in other words it is composed of at least 90% by weight alkyl monoglucoside and alkyl diglucoside molecules, preferably at least 95% by weight alkyl monoglucoside and alkyl diglucoside molecules, more preferably at least 97% by weight alkyl monoglucoside and alkyl diglucoside molecules.

Advantageously, the remainder can be composed of alkyl oligoglucoside molecules, i.e. alkyl glucosides for which the glucoside comprises 3 to 8 glucosyl units, preferably 3 to 5 glucosyl units.

In an embodiment, the alkyl polyglucoside of formula (I) is essentially composed of a mixture of alkyl polyglucoside molecules of formula (I), said mixture advantageously having an average degree of polymerisation between 3 and 25, preferably between 5 and 25, preferably between 6 and 25, preferably between 7 and 25, more preferably between 8 and 25.

The mixture is essentially composed of alkyl polyglucoside molecules of formula (I), in other words it is preferably composed of at least 90% by weight of said alkyl polyglucosides molecules, preferably at least 95% by weight of said alkyl polyglucosides molecules, more preferably at least 97% by weight of said alkyl polyglucosides molecules.

In the present invention, the “average degree of polymerisation” represents the average distribution of the number of glucosyl units per molecule of alkyl polyglucoside of formula (I) or alkyl glucoside of formula (II) within a mixture of alkyl polyglucoside molecules of formula (I) or a mixture of alkyl glucoside molecules of formula (II).

In the invention, this average degree of polymerisation is determined from measurements carried out by nuclear magnetic resonance (NMR).

For a mixture of alkyl polyglucoside molecules of formula (I), the average degree of polymerisation is determined by calculating the ratio (average molecular mass of the mixture alkyl polyglucoside molecules of formula (I)—molar mass of the corresponding alkyl chain): (molar mass of one glucosyl unit).

For a mixture of alkyl glucoside molecules of formula (II), the average degree of polymerisation is determined by calculating the ratio (average molecular mass of the mixture alkyl glucoside molecules of formula (II)—molar mass of the corresponding alkyl chain): (molar mass of one glucosyl unit).

Alkyl Group

Surprisingly, the inventors have discovered that α-transglucosylases of the GH70 family can efficiently glucosylate alkyl glucosides of formula (II) having a large alkyl group.

The alkyl group preferably comprises at least 8, 9, 10, 11, 12, 13, 14, 15, 16, 17, 18 or 19 carbon atoms and at most 20 carbon atoms.

In an embodiment, the alkyl group R advantageously comprises between 8 and 16 carbon atoms, more preferably comprises 8 and 12 carbon atoms.

In an embodiment, the alkyl group R comprises between 9 and 20 carbon atoms, preferably comprises between 9 and 16 carbon atoms, more preferably comprises between 9 and 12 carbon atoms.

In an embodiment, the alkyl group R comprises between 10 and 20 carbon atoms, preferably comprises between 10 and 16 carbon atoms, more preferably comprises between 10 and 12 carbon atoms.

In an embodiment, the alkyl group R comprises between 13 and 20 carbon atoms, preferably comprises between 16 and 20 carbon atoms.

More particularly, the inventors have shown that α-transglucosylases of the GH70 family are capable of catalysing the elongation of the glucosidic part of an alkyl glucoside of formula (II) having an alkyl group R comprising between 8 and 20 carbon atoms, in other words an acceptor substrate having an alkyl pole that is much more hydrophobic than alkyl glucosides having an alkyl group such as a methyl, ethyl, propyl or butyl group.

In other words, the method of the invention is particularly advantageous in that it makes it possible to obtain an alkyl polyglucoside of formula (I) having an alkyl group R of between 8 and 20 carbon atoms and a glucoside part [Glc]ₘ-[Glc]ₙ having n+m as defined above.

In an embodiment, R represents an alkyl group comprising 8 to 12 carbon atoms, and n+m is between 7 and 200, preferably between 8 and 200, preferably between 9 and 200, more preferably between 10 and 200. In this embodiment, n+m can advantageously be between 7 and 50, preferably between 8 and 50, preferably between 9 and 50, and more preferably between 10 and 50.

In an embodiment, R represents an alkyl group comprising 12 to 20 carbon atoms, and n+m is between 3 and 200, preferably between 4 and 200, preferably between 5 and 200, and more preferably between 10 and 200. In this embodiment, n+m can advantageously be between 3 and 50, preferably between 4 and 50, preferably between 5 and 50, and more preferably between 10 and 50.

In an embodiment, when R represents an alkyl group comprising 8 to 12 carbon atoms, n+m is between 7 and 200, preferably between 8 and 200, preferably between 9 and 200, more preferably between 10 and 200 and can, in particular, be between 7 and 50, preferably between 8 and 50, preferably between 9 and 50, and more preferably between 10 and 50; and when R represents an alkyl group comprising 12 to 20 carbon atoms, n+m is between 3 and 200, preferably between 4 and 200, preferably between 5 and 200, and more preferably between 10 and 200 and can, in particular, be between 3 and 50, preferably between 4 and 50, preferably between 5 and 50, and more preferably between 10 and 50.

α-Transglucosylase

According to the invention, the term “α-transglucosylase” designates an enzyme capable of polymerising glucosyl units by a bonds, by catalysing the transfer of a glucosyl unit from a glucosyl sugar donor to an acceptor compound

According to the invention, the expression “of the GH70 family”, relating to α-transglucosylase, means that the α-transglucosylase according to the invention belongs to family 70 of the glycoside hydrolases, according to the CAZy classification CAZy, “Carbohydrate-Active enZYmes” (abbreviated “CAZy”), is a bioinformatics database for classification of enzymes that are active on sugars i.e. capable of catalysing their dissociation or their synthesis according to homologies of sequences or structure, in particular of their catalytic modules and glucoside bonds. In the CAZy classification, the group of glycoside-hydrolases (GH) comprises 167 families composed of enzymes active on sugars capable of catalysing hydrolysis reactions of glycosidic or transglucosylation bonds.

The α-transglucosylases of the GH70 family are typically produced naturally by lactic bacteria of the genera Streptococcus, Leuconostoc (abbreviated “L.”), Weisella or Lactobacillus (abbreviated “Lb.”).

The α-transglucosylases of the GH70 family according to the invention are active on sucrose, which means that α-transglucosylases according to the invention specifically use sucrose or one of its analogues as glucosyl donor.

The analogues of sucrose can be chosen, in particular, from the group comprising α-D-Glucopyranosyl fluoride, Lactulosucrose, p-nitrophenyl α-D-glucopyranoside, α-D-glucopyranosyl α-L-sorbofuranoside and the mixtures thereof.

According to an embodiment, the α-transglucosylase of the GH70 family is selected from one of the α-transglucosylases described below.

The enzyme DSR-M Δ1 with amino acid sequence SEQ ID NO: 1 is a fragment extending from the amino acid at position 42 to the amino acid at position 1433 of the amino acid sequence of the wild-type of the enzyme DSR-M (originating from the strain L. citreum NRRL B-1299) having reference GenBank CDX668951.1.

The enzyme DSR-M Δ5 of amino acid sequence SEQ ID NO: 2 is a fragment extending from the amino acid at position 421 to the amino acid at position 1315 of the amino acid sequence of the wild-type of the enzyme DSR-M (originating from the strain L. citreum NRRL B-1299) having reference GenBank CDX668951.1.

The enzyme DSR-M Δ5 W624A of amino acid sequence SEQ ID NO: 3 is a mutant at position 624 of the fragment of amino acid sequence SEQ ID NO:2.

The enzyme GS-A of amino acid sequence SEQ ID NO: 4 corresponds to the amino acid sequence of the wild-type of the enzyme originating from the strain Lb. kunkeei MP2 having reference GenBank ALJ31412.

The enzyme GS-B of amino acid sequence SEQ ID NO: 5 corresponds to the amino acid sequence of the wild-type of the enzyme originating from the strain Lb. animalis DSM 20602 having reference GenBank KRM57462.1.

The enzyme GS-C of amino acid sequence SEQ ID NO: 6 corresponds to the amino acid sequence of the wild-type of the enzyme originating from the strain Lb. apodemi having reference GenBank WP_056957205.

The enzyme GS-D of amino acid sequence SEQ ID NO: 7 corresponds to the amino acid sequence of the wild-type of the enzyme originating from the strain Lb. animalis DSM 20602 having reference GenBank KRM57463.

The enzyme GS-E of amino acid sequence SEQ ID NO: 8 corresponds to the amino acid sequence of the wild-type of the enzyme originating from the strain Lb. capillatus DSM 19910 having reference GenBank KRL03580.

The enzyme GS-F Δ1 of amino acid sequence SEQ ID NO: 9 is a fragment extending from the amino acid at position 166 to the amino acid at position 1874 of the amino acid sequence of the wild-type of the enzyme originating from the strain L. fallax KCTC 3537 having reference GenBank WP_010006777.1.

The enzyme GS-FS of amino acid sequence SEQ ID NO: 10 corresponds to the amino acid sequence of the wild-type of the enzyme originating from Streptococcus salivarus HSISS4 having reference GenBank ALR80278.

The enzyme DSR-G of amino acid sequence SEQ ID NO: 11 corresponds to the amino acid sequence of the wild-type of the enzyme originating from Lb. kunkeei DSM 12361 having reference GenBank KRK22577.1.

The enzyme DSR-G CD1 of amino acid sequence SEQ ID NO: 12 is a fragment extending from the amino acid at position 1 to the amino acid at position 1407 of the amino acid sequence of the wild-type of the enzyme originating from the strain Lb. kunkeei DSM 12361 having reference GenBank KRK22577.

The enzyme ASR Δ1 of amino acid sequence SEQ ID NO: 13 is a fragment extending from the amino acid at position 1 to the amino acid at position 1425 of the amino acid sequence of the wild-type of the enzyme originating from the strain L. mesenteroides NRRL B-1355 having reference GenBank CAB65910.2.

The enzyme GTF-SI of amino acid sequence SEQ ID NO: 14 corresponds to the amino acid sequence of the wild-type of the enzyme originating from the strain Streptococcus mutans having reference GenBank BAA26114.1.

The enzyme GTF-J of amino acid sequence SEQ ID NO: 15 corresponds to the amino acid sequence of the wild-type of the enzyme originating from the strain Streptococcus mutans having reference GenBank AAA26896.1.

The enzyme GBDCD2 of amino acid sequence SEQ ID NO: 16 is a fragment extending from the amino acid at position 1758 to the amino acid at position 2862 of the amino acid sequence of the wild-type of the enzyme originating from the strain L. citreum NRRL B-1299 having reference GenBank CDX66820.1.

The enzyme BRS-A of amino acid sequence SEQ ID NO: 17 corresponds to the amino acid sequence of the wild-type of the enzyme originating from the strain L. citreum NRRL B-1299 having reference GenBank CDX66896.1.

The enzyme BRS-B Δ1 of amino acid sequence SEQ ID NO: 18 is a fragment extending from the amino acid at position 39 to the amino acid at position 1313 of the amino acid sequence of the wild-type of the enzyme originating from the strain L. citreum NRRL B-742 having reference GenBank CDX65123.1.

The enzyme BRS-C of amino acid sequence SEQ ID NO: 19 corresponds to the amino acid sequence of the wild-type of the enzyme originating from the strain L. fallax KCTC 3537 having reference GenBank ZP_08312597.1.

The enzyme BRS-D Δ1 of amino acid sequence SEQ ID NO: 20 is a fragment extending from the amino acid at position 88 to the amino acid at position 1453 of the amino acid sequence of the wild-type of the enzyme originating from the strain Lb. kunkei EFB6 having reference GenBank WP_051592287.

The enzyme BRS-E Δ1 of amino acid sequence SEQ ID NO: 21 is a fragment extending from the amino acid at position 32 to the amino acid at position 1264 of the amino acid sequence of the wild-type of the enzyme originating from the strain L. mesenteroides KFRI-MG having reference GenBank AHF19404.1.

The enzyme BRS-F of amino acid sequence SEQ ID NO: 22 corresponds to the amino acid sequence of the wild-type of the enzyme originating from the strain Fructobacillus tropaeoli having reference GenBank GAP05007.1.

The enzyme DSR-G CD2 of amino acid sequence SEQ ID NO: 23 is a fragment extending from the amino acid at position 928 to the amino acid at position 2621 of the amino acid sequence of the wild-type of the enzyme originating from the strain Lb. kunkeei DSM 12361 having reference GenBank KRK22577.1.

The α-transglucosylases of the GH70 family according to the invention can be obtained according to methods known to a person skilled in the art, in particular by the method consisting of cultivating a cell that naturally expresses α-transglucosylase or a host cell comprising a transgene coding for α-transglucosylase and expressing said α-transglucosylase, and extracting said α-transglucosylase from these cells or from the culture medium in which the α-transglucosylase has been secreted.

Strains of recombinant bacteria, for example strains of Bacillus subtillis or Lactobacillus, secreting said α-transglucosylases of the GH70 family can be used.

It is also possible to obtain α-transglucosylases of the GH70 family by means of cell free protein expression systems.

In the method of the invention, the α-transglucosylase of the GH70 family is preferably a glucansucrase of the GH70 family, a branching sucrase of the GH70 family, or a mixture thereof.

In the present document, the expression “glucansucrase of the GH70 family”, sometimes abbreviated to “GS”, refers to an α-transglucosylase of the GH70 family that is capable of catalysing the synthesis of α-glucans i.e. polysaccharides composed exclusively of glucosyl units bonded together by alpha bonds. The glucansucrases include dextransucrases (sometimes abbreviated DSR), which synthesise dextrans, i.e. glucans for which the residues of the main chain are mostly alpha-1,6 bonded; the reuteransucrases, which synthesises reuterans, i.e. glucans for which the residues of the main chain are alpha-1,4 and alpha-1,6 bonded; the mutansucrases, which synthesise mutans, i.e. glucans for which the residues of the main chain are mostly alpha-1,3 bonded; alternansucrase, i.e. glucans for which the residues of the main chain are alternately alpha-1,3 and alpha-1,6 bonded. Preferably, the α-transglucosylase of the GH70 family is not an alternansucrase.

In the present document, the terms “branching sucrase of the GH70 family” (sometimes abbreviated BRS) or “α-transglucosylase of the GH70 family with branching activity” or “branching enzyme of the GH70 family” are used interchangeably and refer to an α-transglucosylase of the GH70 family capable of catalysing the addition of glucosyl units in the main chain of a pre-existing dextran-type glucan forming branchings (or otherwise termed branches). The nature of the branching bond (alpha-1,2; alpha-1,3; alpha-1,4 or alpha-1,6 bonds) and the length of the chains of glucosyl units constituting the branchings varies according to the specific properties of the branching sucrase considered.

Preferably, the branching sucrases of the GH70 family according to the invention catalyse branches comprising preferably 1 to 3 glucosyl units, preferably 1 or 2 glucosyl units along alpha-1,2 bonds and/or alpha-1,3 bonds.

Hence, in an embodiment, the glucoside part of the alkyl polyglucoside of formula (I) has the form of a linear chain comprising branches having 1 to 3 glucosyl units, preferably 1 or 2 glucosyl units, said branches being bonded to the linear chain along alpha-1,2 bonds and/or alpha-1,3 bonds.

The inventors have shown that, surprisingly, certain α-transglucosylases of the GH70 family are able to transfer glucosyl units onto the glucoside part from an alkyl glucoside of formula (II) as described above via a glucoside elongation mechanism. According to an embodiment, the α-transglucosylase of the GH70 family is a glucansucrase of the GH70 family.

When the α-transglucosylase of the GH70 family is a glucansucrase of the GH70 family, an alkyl polyglucoside is advantageously obtained for which the glucoside part comprises at least 50%, preferably 60%, more preferably 80% bonds chosen from the group comprising alpha-1,6 bonds and alpha-1,3 bonds, the remainder of the bonds being advantageously alpha-1,4 bonds.

In addition, the glucansucrases of the GH70 family according to the invention make it possible to obtain an alkyl polyglucoside having a long glucoside chain, with n+m as defined above.

The glucansucrase of the GH70 family of the invention preferably has, for amino acid sequence, a sequence chosen from the group comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15.

The inventors have shown, for the first time, that the glucansucrases of the GH70 family of the invention are capable of extending alkyl glucosides of formula (II) by more than 3 glucosyl units starting from sucrose.

Advantageously, when the glucansucrase of the GH70 family according to the invention has, as amino acid sequence, a sequence chosen from the group comprising SEQ ID NO: 1, SEQ ID NO: 2, SEQ ID NO: 3, SEQ ID NO: 5, SEQ ID NO: 6, SEQ ID NO: 8, SEQ ID NO: 9 and SEQ ID NO: 12, an alkyl polyglucoside of formula (I) is obtained for which the glucoside part comprises at least 80% alpha-1,6 bonds, the remainder of the bonds being advantageously alpha-1,3 bonds.

Advantageously, when the glucansucrase of the GH70 family according to the invention has, as amino acid sequence, a sequence chosen from the group comprising SEQ ID NO: 1, SEQ ID NO: 2, SEQ ID NO: 3, SEQ ID NO: 5, SEQ ID NO: 6, and SEQ ID NO: 12, an alkyl polyglucoside of formula (I) is obtained for which the glucoside part comprises at least 90%, preferably at least 95% alpha-1,6 bonds, the remainder of the bonds being advantageously alpha-1,3 bonds.

Advantageously, when the glucansucrase of the GH70 family according to the invention has, as amino acid sequence, a sequence chosen from the group comprising SEQ ID NO: 10, SEQ ID NO: 14, and SEQ ID NO: 15, an alkyl polyglucoside of formula (I) is obtained for which the glucoside part comprises at least 90% alpha-1,3 bonds, the remainder of the bonds being advantageously alpha-1,6 bonds.

Advantageously, when the glucansucrase of the GH70 family according to the invention has, as amino acid sequence, a sequence chosen from the group comprising SEQ ID NO: 8 and SEQ ID NO:9, an alkyl polyglucoside of formula (I) is obtained for which the glucoside part comprises at least 70% alpha-1,6 bonds, the remainder of the bonds being advantageously alpha-1,3 bonds.

Advantageously, when the glucansucrase of the GH70 family according to the invention has, as amino acid sequence, a sequence chosen from the group comprising SEQ ID NO: 4, an alkyl polyglucoside of formula (I) is obtained for which the glucoside part comprises at least 50% alpha-1,6 bonds, the remainder of the bonds being advantageously alpha-1,4 bonds.

According to another embodiment, the α-transglucosylase of the GH70 family according to the invention is a branching sucrase GH70.

When the α-transglucosylase of the GH70 family is a branching sucrase, advantageously an alkyl polyglucoside of formula (I) is obtained for which the glucoside part comprises at least 50%, preferably 60%, more preferably 80% of bonds in the group comprising alpha-1,2, alpha-1,3 bonds and the mixtures thereof; the remainder of the bonds being advantageously alpha-1,6 bonds.

The branching sucrase of the GH70 family according to the invention preferably has, as amino acid sequence, a sequence chosen from the group comprising SEQ ID NO: 16, SEQ ID NO: 17, SEQ ID NO: 18, SEQ ID NO: 19, SEQ ID NO: 20, SEQ ID NO: 21, SEQ ID NO: 22, and SEQ ID NO: 23.

Advantageously, when the branching sucrase of the GH70 family has, as amino acid sequence, a sequence chosen from the group comprising SEQ ID NO: 16, SEQ ID NO: 17, SEQ ID NO: 20, and SEQ ID NO: 22 an alkyl polyglucoside of formula (I) is obtained for which the glucoside part comprises at least 80% alpha-1,2 bonds, preferably 100% alpha-1,2 bonds.

Advantageously, when the branching sucrase GH70 has, as amino acid sequence, a sequence chosen from the group comprising SEQ ID NO: 18, SEQ ID NO: 19, and SEQ ID NO: 23 an alkyl polyglucoside of formula (I) is obtained for which the glucoside part comprises at least 80%, preferably 90%, more preferably 95%, more preferably 98% alpha-1,3 bonds, more preferably 100% alpha-1,3 bonds.

Advantageously, when the branching sucrase GH70 has, as amino acid sequence, a sequence chosen from the group comprising SEQ ID NO: 21, an alkyl polyglucoside of formula (I) is obtained for which the glucoside part comprises at least 60% alpha-1,2 bonds, the remainder of the bonds being advantageously alpha-1,6 bonds.

Implementation of the Method

Step i) can be carried out by simultaneously or successively placing the alkyl glucoside of formula (II) in contact with a plurality of α-transglucosylases of the GH70 family.

In an embodiment, step i) comprises a sub-step i₀) of placing the alkyl glucoside of formula (II) in contact with a mixture of one or more glucansucrases GH70 and one or more GH70 branching enzymes.

This sub-step i₀) preferably uses a ratio of (glucansucrase):(branching sucrase), expressed in units of enzyme activity, of between 0.01 and 10, the activities of each of the enzymes used, advantageously varying between 0.2 and 2 U·ml⁻¹.

Without wishing to be bound by a particular theory, the inventors consider that placing an alkyl glucoside of formula (II) in contact with a mixture of glucansucrase(s) and branching sucrase(s) according to the invention during step i₀) promotes the initiation of the elongation reaction.

In an embodiment, step i) comprises a step i₁) placing the alkyl glucoside of formula (II) in contact with one or more glucansucrases of the GH70 family.

Without wishing to be bound by a theory, the inventors consider that this embodiment initially enables promoting of the elongation of the glucoside part in the form of a long linear chain.

In an embodiment, step i) comprises placing the alkyl glucoside of formula (II) in contact with one or more glucansucrases of the GH70 family then a step i₂) of placing the alkyl glucoside obtained at the end of step i₁) in contact with one or more branching sucrases of the GH70 family.

Without wishing to be bound by a theory, the inventors consider that this embodiment initially promotes the elongation of the glucoside part in the form of a long linear chain, then secondly promotes the elongation of the glucoside part in the form of branches.

This embodiment also has the advantage of increasing the diversity of chemical structures obtained.

Indeed, the use of branching sucrases of the GH70 family according to the invention make it possible to control the number of branchings introduced, according to the reaction conditions, so as to obtain an alkyl polyglucoside of formula (I) for which the glucoside part comprises at least 50% alpha-1,6 bonds, the remainder of the glucosidic bonds being alpha-1,3 bonds and/or alpha-1,2 bonds, the sum of the percentages of alpha-1,6 bonds, percentages of alpha-1,3 bonds and percentages of alpha-1,2 bonds being equal to 100%.

For example, it is possible to obtain an alkyl polyglucoside of formula (I) for which the glucoside part comprises at least 60% alpha-1,6 bonds, at least 2% alpha-1,3 bonds and at least 2% alpha-1,6 bonds, preferably between 2% and 35% alpha-1,3 bonds and at least 1% alpha-1,2 bonds, preferably between 2% and 35% alpha-1,2 bonds, the sum of the percentages of alpha-1,6 bonds, percentages of alpha-1,3 bonds and percentages of alpha-1,2 bonds being equal to 100%.

In a particular embodiment, step i) is performed in solution at a controlled pH, in particular through the use of a buffer solution.

Step i) is advantageously carried out at a pH value between 5 and 8.

Step i) is advantageously carried out with an initial concentration of sucrose or of a sucrose analogue of between 20 and 660 g·L⁻¹.

Step i) is preferably performed with a molar ratio of alkyl polyglucoside of formula (II):sucrose or sucrose analogue of between 0.001 and 10, preferably between 0.001 and 5, preferably 0.001 and 0.3, preferably between 0.002 and 0.006.

Importantly, the inventors have shown that the elongation of the chain can be modulated as a function of the molar ratio of polyglucoside of formula (II):sucrose or sucrose analogue. Without wishing to be bound by a particular theory, the inventors consider that the higher the molar ratio of alkyl polyglucosides of formula (II):sucrose or sucrose analogue, the less sucrose is available to be a donor and therefore the less the acceptor is extended.

Advantageously, step i) is performed with a molar ratio of alkyl polyglucosides of formula (II):sucrose or sucrose analogue between 0.05 and 5. Step i) is preferably implemented at a temperature between 10° C. and 80° C.

Preferably, step i) is carried out with α-transglucosylase of the GH70 family in solid form, in solution, in suspension or immobilised.

The method may in addition advantageously comprise a step ii) of purifying the alkyl polyglucoside of formula (I) obtained at the end of step i).

Alkyl Polyglucosides that can be Obtained by the Method According to the Invention

Alkyl polyglucoside of formula (I) obtained by implementing the method as defined by the first object of the invention, in particular at the end of step i), constitutes a second object of the invention.

The invention therefore has, as second object, an alkyl polyglucoside that can be obtained by implementing the method in accordance with the first object of the invention, said alkyl polyglucoside being characterised in that it is of formula (I):

[Glc]ₘ—[Glc]ₙ(-O—R) (I)

in which:
R represents a linear or branched, saturated or unsaturated alkyl group, comprising between 8 and 20 carbon atoms,
[Glc]ₘ-[Glc]ₙ represents a linear or branched glucoside part comprising n+m glucosyl units, n+m being between 3 and 200.

The alkyl polyglucoside of formula (I) that can be obtained by implementing the method according to the first object of the invention can have a wide structural diversity, in particular in its glucoside part, in particular in terms of size, structure (branched or otherwise) and nature of the osidic bonds.

Hence, the alkyl polyglucoside of formula (I) of the second object of the invention is as defined in detailed manner in the first object of the invention. In particular, the number of carbon atoms of the alkyl group R, the number n+m glucosyl units within the glucoside part [Glc]ₘ-[Glc]ₙ, as well as the nature of the bonds within the fractions of the glucoside part [Glc]ₘ-[Glc]ₙ and that of the bond between the alkyl group R and the glucoside fraction [Glc], are as previously mentioned.

In an embodiment, the alkyl polyglucoside of formula (I) that can be obtained by implementing the method in accordance with the first object of the invention, is an alkyl polyglucoside of formula (I), wherein R represents an alkyl group comprising 8 to 12 carbon atoms and wherein the number n+m is between 7 and 200, preferably between 8 and 200, preferably between 9 and 200, more preferably between 10 and 200. In this embodiment, n+m can, in particular, be between 7 and 50, preferably between 8 and 50, preferably between 9 and 50, and more preferably between 10 and 50.

In an embodiment, the alkyl polyglucoside of formula (I) that can be obtained by implementing the method in accordance with the first object of the invention is an alkyl polyglucoside of formula (I), wherein R represents an alkyl group comprising 12 to 20 carbon atoms and wherein n+m is between 3 and 200, preferably between 4 and 200, preferably between 5 and 200, more preferably between 10 and 200. In this embodiment, n+m can, in particular, be between 3 and 50, preferably between 4 and 50, preferably between 5 and 50, and more preferably between 10 and 50.

In an embodiment, the alkyl polyglucoside of formula (I) that can be obtained by the method according to the first object of the invention is an alkyl polyglucoside of formula (I) wherein:
when R represents an alkyl group comprising 8 to 12 carbon atoms, n+m is between 7 and 200, preferably between 8 and 200, preferably between 9 and 200, more preferably between 10 and 200 and n+m can be, in particular, between 7 and 50, preferably between 8 and 50, preferably between 9 and 50, and more preferably between 10 and 50; and
when R represents an alkyl group comprising 12 to 20 carbon atoms, n+m is between 3 and 200, preferably between 4 and 200, preferably between 5 and 200, and more preferably between 10 and 200 and can be, in particular, between 3 and 50, preferably between 4 and 50, preferably between 5 and 50, and more preferably between 10 and 50.

In an embodiment, the alkyl polyglucoside that can be obtained by implementing the method in accordance with the first object of the invention is an alkyl polyglucoside of formula (I) substantially consisting of a mixture of alkyl polyglucoside molecules of formula (I), said mixture having an average degree of polymerisation between 3 and 25, preferably between 5 and 25, preferably between 6 and 25, preferably between 7 and 25, more preferably between 8 and 25. The mixture is essentially composed of alkyl polyglucoside molecules of formula (I), in other words it is composed of at least 90% by weight of said alkyl polyglucosides, preferably at least 95% by weight of said alkyl polyglucosides, more preferably at least 97% by weight of said alkyl polyglucosides.

In an embodiment, the alkyl polyglucoside that can be obtained by implementing the method in accordance with the first object of the invention is an alkyl polyglucoside of formula (I) for which the glucoside part comprises at least 50%, preferably 60%, more preferably 80% alpha-1,6 bonds or alpha-1,3 bonds, the remainder of the bonds being advantageously alpha-1,4 bonds and/or alpha-1,2 bonds.

In an embodiment, the alkyl polyglucoside that can be obtained by implementing the method in accordance with the first object of the invention is an alkyl polyglucoside of formula (I) for which the glucoside part comprises at least 50%, preferably at least 70% alpha-1,6 bonds, the remainder of the bonds being advantageously alpha-1,3 bonds.

In an embodiment, the alkyl polyglucoside that can be obtained by implementing the method in accordance with the first object of the invention is an alkyl polyglucoside of formula (I) for which the glucoside part comprises at least 60% alpha-1,6 bonds, at least 2% alpha-1,3 bonds and at least 2% alpha-1,6 bonds, preferably between 2% and 35% alpha-1,3 bonds and at least 1% alpha-1,2 bonds, preferably between 2% and 35% alpha-1,2 bonds, the sum of the percentages alpha-1,6 bonds, percentages of alpha-1,3 bonds and percentages of alpha-1,2 bonds being equal to 100%.

In an embodiment, the alkyl polyglucoside that can be obtained by implementing the method in accordance with the first object of the invention is an alkyl polyglucoside of formula (I) for which the glucoside part comprises at least 90% alpha-1,3 bonds, the remainder of the bonds being advantageously alpha-1,6 bonds.

In an embodiment, the alkyl polyglucoside that can be obtained by implementing the method in accordance with the first object of the invention is an alkyl polyglucoside of formula (I) for which the glucoside part comprises at least 50% alpha-1,6 bonds, the remainder of the bonds being advantageously alpha-1,4 bonds.

In an embodiment, the alkyl polyglucoside that can be obtained by implementing the method in accordance with the first object of the invention is an alkyl polyglucoside of formula (I) for which the glucoside part comprises at least 70% alpha-1,6 bonds, the remainder of the bonds being advantageously alpha-1,3 bonds.

In an embodiment, the alkyl polyglucoside that can be obtained by implementing the method in accordance with the first object of the invention is an alkyl polyglucoside of formula (I) for which the glucoside part comprises at least 80% alpha-1,2 bonds, the remainder of the bonds being advantageously alpha-1,3 bonds.

In an embodiment, the alkyl polyglucoside that can be obtained by implementing the method in accordance with the first object of the invention is an alkyl polyglucoside of formula (I) for which the glucoside part comprises at least 80%, preferably 90%, more preferably 95%, more preferably 98%, alpha-1,3 bonds, the remainder of the bonds being advantageously alpha-1,2 bonds.

In an embodiment, the alkyl polyglucoside that can be obtained by implementing the method in accordance with the first object of the invention is an alkyl polyglucoside of formula (I) for which the glucoside part comprises at least 80%, preferably 90%, more preferably 95%, more preferably 98%, alpha-1,3 bonds, the remainder of the bonds being advantageously alpha-1,2 bonds.

In an embodiment, the alkyl polyglucoside that can be obtained by implementing the method in accordance with the first object of the invention is an alkyl polyglucoside of formula (I) for which the glucoside part comprises at least 60% alpha-1,2 bonds, the remainder of the bonds being advantageously alpha-1,6 bonds.

Use of the Alkyl Polyglucoside that can be Obtained by the Method According to the Invention

A third object of the invention is the use of an alkyl polyglucoside that can be obtained by the method according to the invention or as defined in the second object of the invention, as a surfactant.

### Use of α-Transglucosylases According to the Invention for the Glucoside Elongation of an Alkyl Glucoside.

A fourth object of the invention is the use of a α-transglucosylase of the GH70 family as defined in the first object of the invention, in particular a glucansucrase of the GH70 family or a branching sucrase of the GH70 family as defined in the first object of the invention, for the glucoside elongation of an alkyl glucoside, in particular an alkyl glucoside of formula (II) as defined in the first object of the invention.

The present invention is illustrated by the following exemplary embodiments, by which it is not however limited.

### EXAMPLES

### Example 1: Enzymes, Organisms of Origin and Specification

The enzymes used are listed in Table 1.

The sequence SEQ ID NO:3 corresponds to the mutant of the enzyme DSR-M Δ5 at position 624 where the wild tryptophan is replaced by an alanine.

TABLE 1
α-transglucosylases GH70 and bond specifications during
synthesis of the natural polymer. P: polymerase i.e. glucansucrase
GH70; B: branching sucrase GH70; Bif: Bifunctional (enzyme
having two catalytic domains); nd = not determined.

| SEQ ID | | Reaction specification | | | | |
|---|---|---|---|---|---|---|
| NO: | Name | B/P | α-1.2 | α -1.3 | α -1.6 | α-1.4 |
| 1 | DSR-M Δ1 | P | — | — | 100% | — |
| 2 | DSR-M Δ5 | P | — | — | 100% | — |
| 3 | DSR-M Δ5 | P | — | — | 100% | — |
| | W624A | | | | | |
| 4 | GS-A | P | | | 56% | 44% |
| 5 | GS-B | P | | | 100% | |
| 6 | GS-C | P | | | 100% | |
| 7 | GS-D | P | | | | |
| 8 | GS-E | P | | 20% | 80% | |
| 9 | GS-F Δ1 | P | — | 18% | 82% | — |
| 10 | GS-FS | P | — | 100% | — | — |
| 11 | DSR-G | Bif | — | 37% | 63% | — |
| 12 | DSR-G CD1 | P | — | — | 100% | — |
| 13 | ASR Δ1 | P | — | 55% | 45% | — |
| 14 | GTF-SI | P | — | 100% | — | — |
| 15 | GTF-J | P | — | 95% | 5% | — |
| 16 | GBD-CD2 | B | 100% | — | — | — |
| 17 | BRS-A | B | 100% | — | — | |
| 18 | BRS-B Δ1 | B | — | 100% | — | — |
| 19 | BRS-C | B | — | 100% | — | — |
| 20 | BRS-D Δ1 | B | 100% | — | — | — |
| 21 | BRS-E Δ1 | B | 100% | — | — | — |
| 22 | BRS-F | B | 100% | — | — | — |
| 23 | DSR-G CD2 | B | — | 100% | — | — |

### Example 2: Heterologous Expression of Enzymes in Escherichia coli

The genes coding for the enzymes cited above are cloned in vectors enabling recombinant expression in E. coli, under the control of pBad or pET promoters.

The recombinant enzymes are produced by cells of E. coli BL21 DE3 Al or BL21 DE3 Star transformed with the plasmid containing the gene of the targeted enzymes (see Table 2).

TABLE 2
plasmids of the enzymes used and expression systems suitable for the various
enzymes. (Gly: Glycerol; Glu: Glucose; α-Lac: α-Lactose; L-Ara: L-Arabinose)

| | | | Conditions of the modified | | | |
|---|---|---|---|---|---|---|
| SEQ NO: | | | environment ZYM5052 | | | Activity |
| ID | Enzyme | Plasmid | Gly | Glu | Inducer | buffer |
| 1 | DSR-M Δ1 | pET55 | 1% | 0% | 0.1% α-Lac | Acetate 50 mM |
| | | | | | | pH = 5.75 |
| 2 | DSR-M Δ5 | pET55 | 1% | 0% | 0.1% α-Lac | Acetate 50 mM |
| | | | | | | pH = 5.75 |
| 3 | DSR-M Δ5 | pET55 | 1% | 0% | 0.1% α-Lac | Acetate 50 mM |
| | W624A | | | | | pH = 5.75 |
| 4 | GS-A | pET21a | 0.5% | 0.05% | 0.2% α-Lac | Acetate 50 mM |
| | | | | | | pH = 5.75 |
| 5 | GS-B | pET21a | 0.5% | 0.05% | 0.2% α-Lac | Acetate 50 mM |
| | | | | | | pH = 5.75 |
| 6 | GS-C | pET21a | 0.5% | 0.05% | 0.2% α-Lac | Acetate 50 mM |
| | | | | | | pH = 5.75 |
| 7 | GS-D | pET21a | 0.5% | 0.05% | 0.2% α-Lac | Acetate 50 mM |
| | | | | | | pH = 5.75 |
| 8 | GS-E | pET21a | 0.5% | 0.05% | 0.2% α-Lac | Acetate 50 mM |
| | | | | | | pH = 5.75 |
| 9 | GS-F Δ1 | pET53 | 1% | 0% | 1% α-Lac | Acetate 50 mM |
| | | | | | | pH = 5.75 |
| 10 | GS-FS | pET21a | 0.5% | 0.05% | 0.2% α-Lac | Acetate 50 mM |
| | | | | | | pH = 5.75 |
| 11 | DSR-G | pET53 | 1% | 0% | 1% α-Lac | Acetate 50 mM |
| | | | | | | pH = 5.75 |
| 12 | DSR-G CD1 | pET53 | 1% | 0% | 1% α-Lac | Acetate 50 mM |
| | | | | | | pH = 5.75 |
| 13 | ASR Δ1 | pBAD49 | 0.5% | 0.05% | 0.01% L-Ara | Acetate 50 mM |
| | | | | | | pH = 5.2 |
| 14 | GTF-SI | pET21a(+) | 1% | 0% | 1% α-Lac | Phosphate 10 mM |
| | | | | | | pH = 6.5 |
| 15 | GTF-J | pET21a(+) | 1% | 0% | 1% α-Lac | Phosphate 10 mM |
| | | | | | | pH = 6.5 |
| 16 | GBD-CD2 | pET53 | 1.5% | 0.05% | 1% α-Lac | Acetate 50 mM |
| | | | | | | pH = 5.75 |
| 17 | BRS-A | pBAD49 | 0.5% | 0.05% | 0.01% L-Ara | Acetate 50 mM |
| | | | | | | pH = 5.75 |
| 18 | BRS-B Δ1 | pET55 | 1% | 0% | 0.1% α-Lac | Acetate 50 mM |
| | | | | | | pH = 5.75 |
| 19 | BRS-C | pET55 | 1% | 0% | 0.1% α-Lac | Acetate 50 nM |
| | | | | | | pH = 5.75 |
| 20 | BRS-D Δ1 | pET53 | 1% | 0% | 1% α-Lac | Acetate 50 mM |
| | | | | | | pH = 5.75 |
| 21 | BRS-E Δ1 | pET60 | 1% | 0% | 0.1% α-Lac | Acetate 50 nM |
| | | | | | | pH = 5.75 |
| 22 | BRS-F | pET21a | 0.5% | 0.05% | 0.2% α-Lac | Acetate 50 mM |
| | | | | | | pH = 5.75 |
| 23 | DSR-G CD2 | pET53 | 1% | 0% | 1% α-Lac | Acetate 50 mM |
| | | | | | | pH = 5.75 |

Three hundred microlitres of the transformation mixture enable inoculation of a volume of 30 mL of pre-culture in Lysogeny Broth (LB) medium, supplemented with 100 μg·mL⁻¹ of ampicillin. Each preculture is incubated at 37° C. for 10 hours under stirring at 150 rpm. Cultures of 1 L in modified ZYM5052 medium, the properties of which are presented in Table 2 are inoculated at an initial optical density (OD) OD_{λ=600 nm} of 0.05 from the previous day's preculture, then incubated for 26 hours at 21° C. and at 150 rpm.

At the end of fermentation, the culture media are centrifuged for 15 minutes at 6500 rpm and at a temperature of 4° C. The cell pellets are concentrated to an OD of 80 in the activity buffer (see Table 2). The cells are the broken up by ultrasound according to the following protocol: 5 cycles of 20 seconds at 30% of the maximum power of the probe, cold (ice bath), spaced apart by 4 minutes resting in the ice. The sonication supernatants containing the soluble enzymes of interest are then recovered after 30 minutes centrifugation (10,000 rpm, 10° C.) and stored at 4° C.

### Example 3: Determination of the Enzymatic Activity by Assay of Reducing Sugars with DNS

The enzymatic activity of glucansucrases and branching sucrases is determined by measuring the initial rate of production of reducing sugars using the dinitrosalicylic acid method (DNS) (Miller, 1959).

One enzymatic unit represents the quantity of enzyme which releases one μmole of fructose per minute, at 30° C., for an initial concentration of sucrose of 100 g·L⁻¹ under suitable activity buffer conditions.

During kinetics of 1 mL volume, 100 μL of reaction medium are drawn off and the reaction is stopped by the addition of an equivalent volume of DNS. The samples are then heated for 5 minutes at 95° C., cooled in ice, diluted to half in water, and the absorbance is measured at 540 nm. A standard range of 0 to 2 g·L⁻¹ of fructose can establish the link between the absorbance value and the concentration of reducing sugars.

Acceptor Reactions

The acceptor reactions are carried out in a volume between 1 mL and 15 mL and for a final concentration of sucrose between 146 and 730 mM. The concentrations of alkyl polyglucosides are dependent on their solubility and vary between:
20 and 50 mM for octyl monoglucoside,
10 and 30 mM for decyl monoglucoside,
1 and 5 mM for dodecyl monoglucoside,
10 and 20 mM for dodecyl diglucoside.

In the case of triton CG110 (commercial mixture of C8 and C 10 APGs), the reaction was carried out in the presence of 2 mg to 10 mg of Triton CG110 per mL of reaction.

The reaction is initiated by the addition of a sufficient volume of cellular lysate in order to obtain an enzymatic activity in reaction of 1 U·mL⁻¹. The reactions were incubated at 37° C. and stirred at 800 rpm. At the end of 24 hours, the enzymes are denatured at 95° C. for 5 minutes. The reactions are stored at −18° C. before analysis of the reaction products by high-performance liquid chromatography (HPLC).

Purification of the Reaction Products

A prepurification step of the glucosylated hydroxy-lipids coming from 11-hydroxy-undecanoic acid is carried out by flash chromatography using a REVELERIS® X2 Flash Chromatography System (GRACE, USA) equipped with an 80 g C18 column. The glucosylation products are separated from the residual free sugars under the following conditions:
flow rate 60 ml·min⁻¹, detection by ELSD
0.5 column volumes (CV) to 100% H₂O
gradient ranging from 0% ultrapure H₂O to 50% acetonitrile in 8.5 column volumes (CV)
1 CV at 50% acetonitrile
return to 100% ultrapure H₂O in 0.2 CV
equilibration at 100% ultrapure H₂O during 1.5 CV

The fractions containing the reaction products eluted between 4 and 10 CV are collected, partially evaporated in the rotary evaporator and then freeze-dried. The samples are stored at ambient temperature and away from humidity.

Analytical Techniques

For the HPLC analysis, the reaction media are diluted to half in absolute ethanol. This dilution enables, among other things, the removal of potential high molecular mass polymers, by precipitation.

The separation of lipid acceptors and their glucosylated forms is performed by reversed-phase chromatography with a Synergi™ Fusion-RP column (porosity 80 Å, particle size 4 μm, C18 grafting with polar end capping, Phenomenex, USA). This column is maintained at 30° C. on a Thermo U3000 HPLC system coupled to a Corona CAD Veo detector (Charged Aerosol Detector) (Thermo Scientific, USA). The atomisation temperature is fixed at 50° C. and the filter adjusted to 3.2 seconds.

The mobile phase is composed of a mixture of ultrapure water (solvent A)/HPLC-quality acetonitrile (solvent B), both containing 0.05% (v/v) formic acid. The elution is carried out at a flow rate of 1 mL·min⁻¹ along the following gradient:
between 0 and 5 minutes, a first elution phase with 0% (v/v) of pathway B enables the removal of residual single sugars (residual fructose, glucose, leucrose, sucrose or any short oligosaccharides);
a second gradient phrase ranging from 0% is carried out at 100% of pathway B for 25 minutes allowing the different glucosylated lipid compounds to be separated;
a last phase of 5 minutes at 100% of pathway B enables the regeneration of the column.

NMR Analysis

The characterisation of the products from glucosylation of the various APG is carried out by NMR. The products are diluted in D20 and in the presence of deuterated sodium trimethylsilyl propanoate (TSP-d4) used as internal reference. The 1H spectra were recorded using a Bruker Avance 500 MHz instrument at 298K with a 5 mm z-gradient TBI probe. The data were acquired and processed using the TopSpin 3 software.

### Example 4: Synthesis of Alkyl Glucosides

4.1. General Protocol

26 α-transglucosylases of the GH70 family have been tested for their suitability to extend alkyl-glycosides of different structures and sizes:
Octyl-β-D-glucoside, C₈G₁
Decyl-β-D-Glucoside, C₁₀G₁,
Dodecyl-β-D-Glucoside, C₁₂G₁
Dodecyl-β-D-maltoside, C₁₂G₂,
Hexadecyl maltoside, C₁₆G₂
Triton CG 110 (DuPont, USA), a mixture containing mostly C₈G₁, C₁₀G₁ and in a minority APGs of higher polymerisation degree.

The enzymes tested are listed in Table 3. They have all been produced in recombinant form and expressed in Escherichia coli.

The enzymatic extracts obtained by fermentation are used raw after breaking up the cells and centrifugation of the cellular debris.

The acceptor reactions using GSs were carried out in a volume of 1 to 10 mL at variable concentrations of APG depending on their solubility in water (between 5 mM for C₁₂G₁ and 30 mM for C₈G₁) and sucrose concentrations varying between 146 mM (50 g·L⁻¹) and 1316 mM (450 g·L⁻¹).

All the reactions were carried out in the presence of 1 U·mL⁻¹ of enzyme, a sodium acetate buffer 50 mM pH=5.75, incubated at 37° C. and under stirring at 800 rpm. At the end of 24 hours, the enzymes are denatured by incubation at 95° C. for 5 minutes. In view of their analyses by HPLC-CAD, the reaction media are diluted to ½ in ethanol and centrifuged for 5 minutes at 11,000 g in order to remove the glucans reaction by-products and the flocculated proteins.

The separation of the various reaction products is performed by reversed-phase chromatography using a Synergi™ Fusion-RP 250 mm×2 mm column (porosity: 80 Å, particle size: 4 μm, Phenomenex, USA). This column is held at 30° C. on a Thermo Ultimate 3000 HPLC system equipped with a Corona Veo detector. The mobile phase is composed of ultrapure water (solvent A)/LC-MS quality acetonitrile (solvent B) mixture, each containing 0.05% (v/v) formic acid. The separation is ensured in 35 minutes by a linear gradient, in solvent B, defined as follows: 0 minutes, 0% (v/v); 5 minutes, 0%; 35 minutes, 100%.

4.2. Results

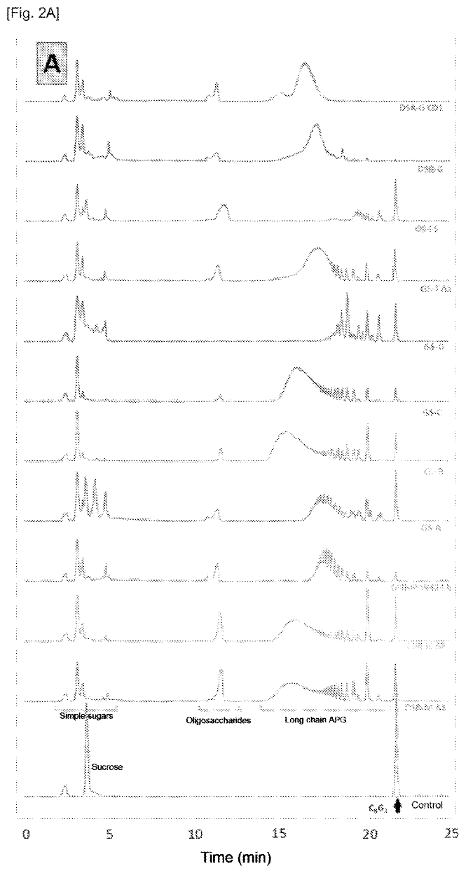

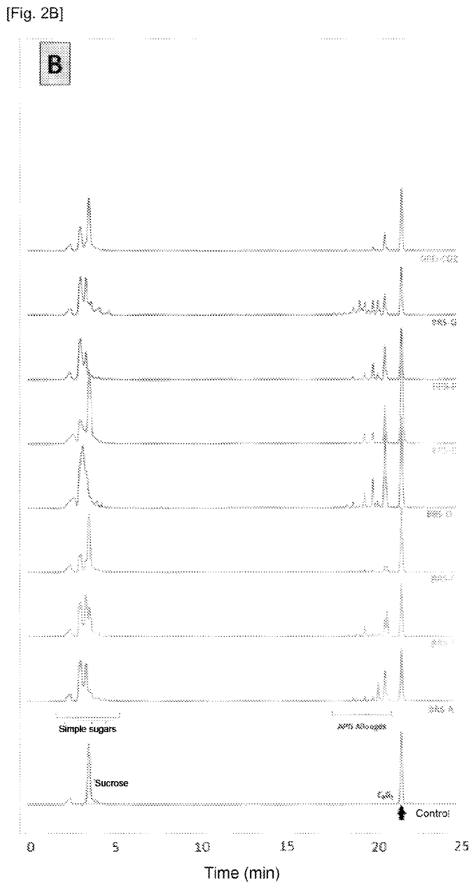

The results obtained during the glucosylation of C₈G₁ by the α-transglucosylases of the sucrose-using GH70 family are presented in FIGS. 2A and 2B.

FIGS. 2A and 2B show the elongation profiles for the substrate C8G1 obtained with α-transglucosylases of the GH70 family which use sucrose as substrate. FIG. 2A shows the profiles obtained with glucansucrases in Chromatogram A: reactions with [C8G1]=30 mM, [sucrose]=585 mM for the glucansucrases DSR-M Δ1 (SEQ ID NO: 1), DSR-M Δ5 (SEQ ID NO: 2), DSR-M Δ5 W624A (SEQ ID NO: 3), GS-B (SEQ ID NO: 5), GS-C(SEQ ID NO: 6), GS-D (SEQ ID NO: 9) and GS-FS Δ1 (SEQ ID NO: 10) and 1170 mM sucrose for the enzymes GS-A SEQ ID NO:4, GS-D (SEQ ID NO:7), DSR-G (SEQ ID NO:11), DSR-G CD1 (SEQ ID NO:12). FIG. 2B shows the profiles obtained with the branching sucrases BRS-A (SEQ ID NO: 17), BRS-B Δ1 (SEQ ID NO: 18), BRS-C(SEQ ID NO: 19), BRS-D Δ1 (SEQ ID NO: 20), BRS-E Δ1 (SEQ ID NO: 21), BRS-F (SEQ ID NO: 22), DSR-G CD2 (SEQ ID NO: 23), GBD-CD2 (SEQ ID NO: 16) shown in chromatogram B, from [C8G1]=20 mM and [sucrose]=585 mM.

All of the tested enzymes enabled elongation of the APG substrate.

It is observed that the profiles of the products obtained are different depending on the class of enzymes used (glucansucrases GH70 and/or branching sucrases GH70).

The branching enzymes (FIG. 2B) enable the addition of 1 to 7 glucosyl units on C₈G₁ depending on the enzymes considered, with conversion rates varying from 20% to 60% under the tested conditions.

With the glucansucrases (FIG. 2A), the sizes of the APG obtained are much more extended, the products carrying a large number of glucosyl units.

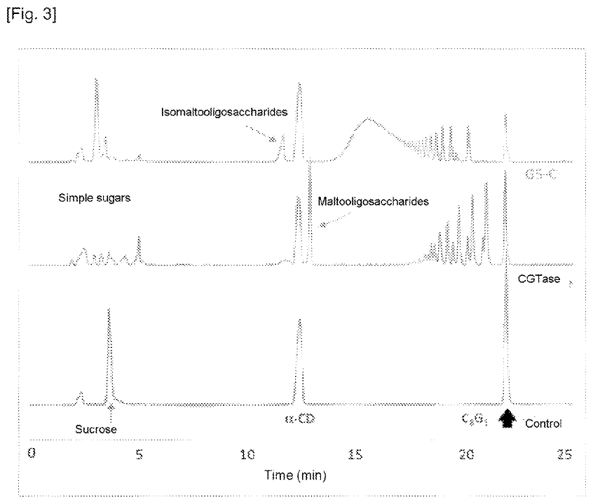

By way of comparison, FIG. 3 shows the elongation profiles of C₈G₁ obtained with i) the glucansucrase GS-C(SEQ ID NO:6) in the presence of sucrose and α-cyclodextrins, and ii) the CGTase of Bacillus macerans (not according to the invention) in the presence of α-cyclodextrins under similar conditions to the work of Svenson and co-workers (Svenson et al., 2009). Profiles clearly show the polymer nature of the glucosidic head synthesised by the glucansucrase DSR-M Δ1 (SEQ ID NO:1) (and all of the glucansucrases tested), in contrast to the oligomeric profile of the APG obtained with CGTase.

Branching Sucrases

Furthermore, the APG produced by glucansucrases can themselves be used as substrates by the branching sucrases.

The latter enable “decoring” of the glucidic heads by glucosyl units α-1,2 or α-1,3 bonded glucosyl units.

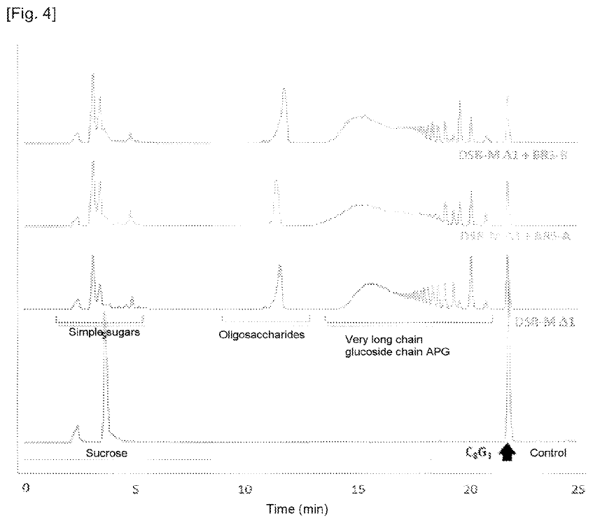

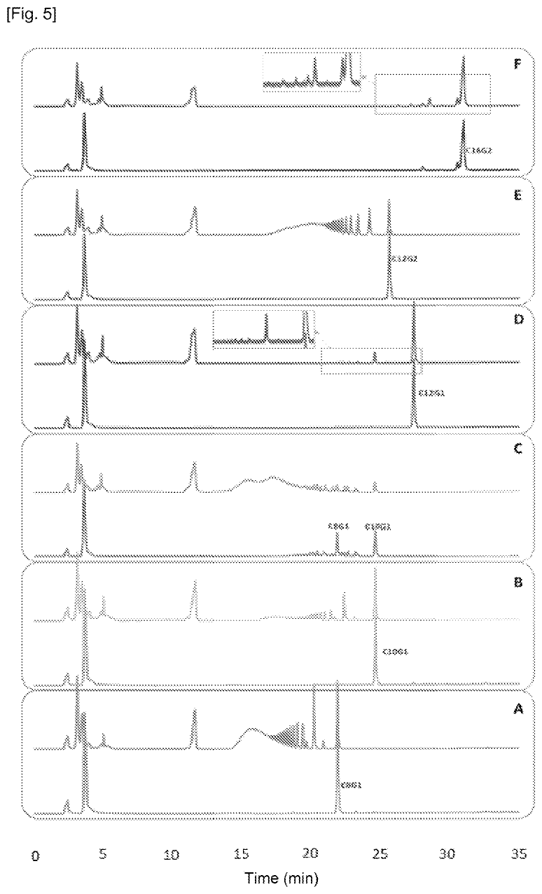

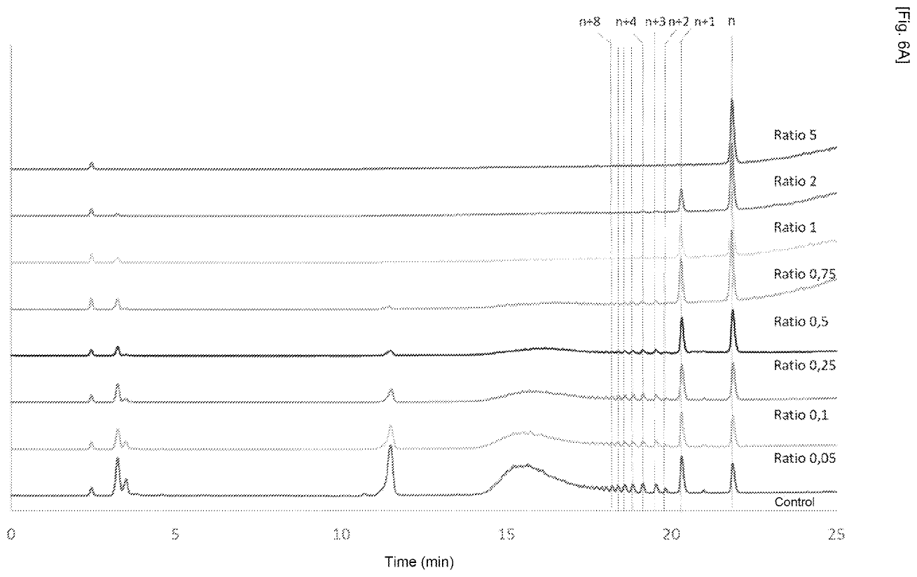

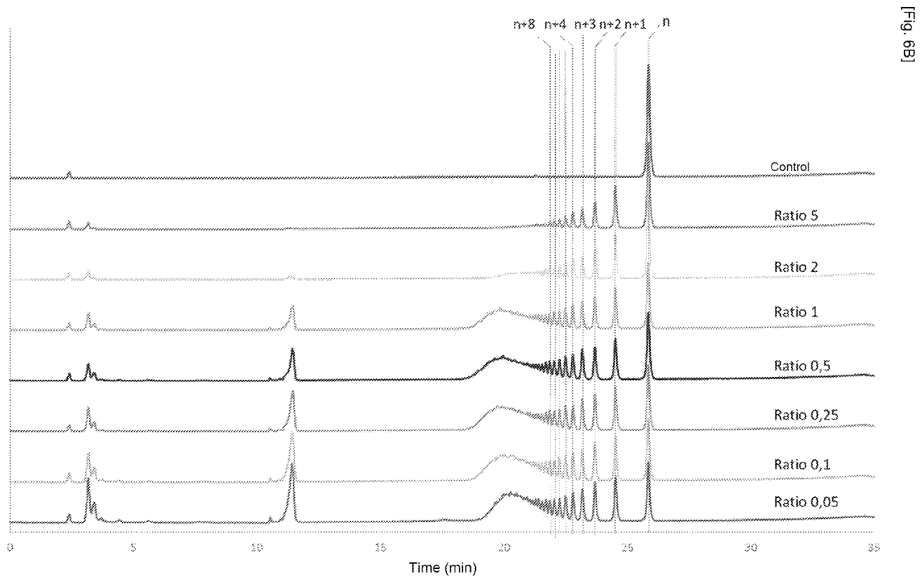

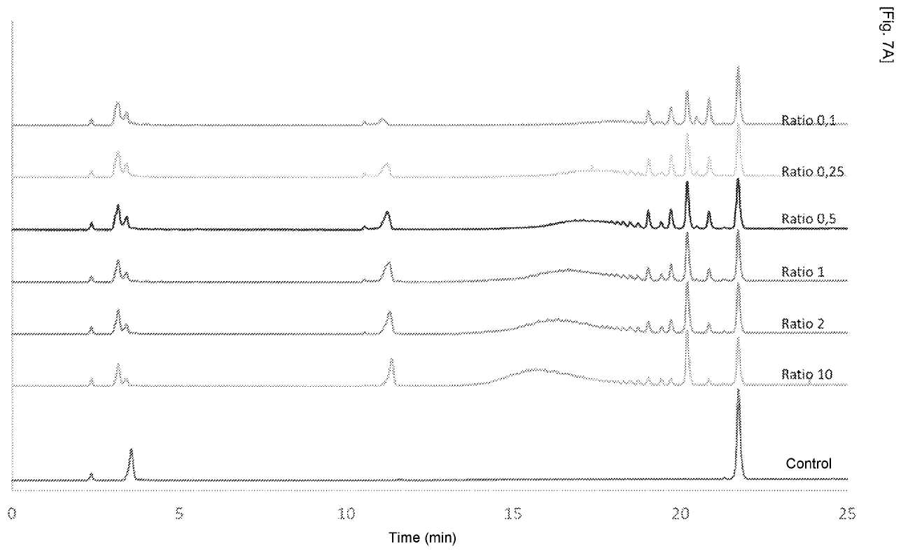

This is illustrated by FIG. 4 where a modification of the profile of the products obtained with glucansucrase DSR-M Δ1 (SEQ ID NO:1) alone is observed compared with the profile of products obtained with branching enzymes BRS-A (SEQ ID NO:17) and BRS-B Δ1 (SEQ ID NO:18) used in a second step after the addition of additional sucrose.

The APG produced by all of the polymerase enzymes were purified and characterised by ¹H NMR, recorded on a 500 MHz BrukerAvance instrument at 298K with a 5 mm z-gradient TBI probe. The data were acquired and processed using the TopSpin3 software. The percentage of each bond is calculated using the relative intensities of anomeric protons of the glucosyl units engaged in a bond with the APG, by integrating the area under the peaks. The average DP is determined by the sum of the relative intensity of the same anomeric protons by taking the proton H1 of the first sugar bonded to the alkyl chain in beta conformation (δ=4.5 ppm) as reference.

The obtained results presented in Table 3 show the possibility of modulating the average DP through the choice of initial concentrations of substrates and the molar ratio (sucrose/APG) and the enzyme used.

TABLE 3
structural characteristics of APG with very long
glucoside heads. Synthesis conditions [C₈G₁]
= 30 mM, [sucrose] = 585 mM for
the enzymes SEQ ID 1, SEQ ID NO: 2, SEQ ID NO:
3, SEQ ID NO: 5, SEQ ID NO: 6, SEQ ID NO: 9, SEQ
ID NO: 10 and 1170 mM for the enzymes SEQ ID NO:
4, SEQ ID NO: 7, SEQ ID NO: 11, SEQ ID NO: 12.

| SEQ ID | | percentage bond | | | | |
|---|---|---|---|---|---|---|
| NO | Enzyme(s) | α-1.2 | α-1.3 | α-1.4 | α-1.6 | DPavr |
| 1 | DSR-M Δ1, | | | | 100% | 10.04 |
| | [sacch] = 200 g/L | | | | | |
| 1 | DSR-M Δ1, | | 4% | | 96% | 11.35 |
| | [sacch] = 400 g/L | | | | | |
| 2 | DSR-M Δ5 | | | | 100% | 8.81 |
| 3 | DSR-M Δ5 W624A | | | | 100% | 9.35 |
| 4 | GS-A | | | 43% | 57% | 5.59 |
| 5 | GS-B | | 1% | | 99% | 17.89 |
| 6 | GS-C | | | | 100% | Nd |
| 7 | GS-D | | 31% | | 69% | 3.97 |
| 9 | GS-F Δ1 | | 12% | | 88% | 9.65 |
| 10 | GS-FS | | 100% | | | 2.24 |
| 11 | DSR-G | | 29% | | 71% | 12.52 |
| 12 | DSR-G CD1 | | 5% | | 95% | 20.22 |
| 1 + 17 | DSR-M Δ1 + | 20% | 3% | | 77% | 11.05 |
| | BRS-A | | | | | |
| 1 + 18 | DSR-M Δ1 + | 2% | 32% | | 66% | 12.48 |
| | BRS-B Δ1 | | | | | |

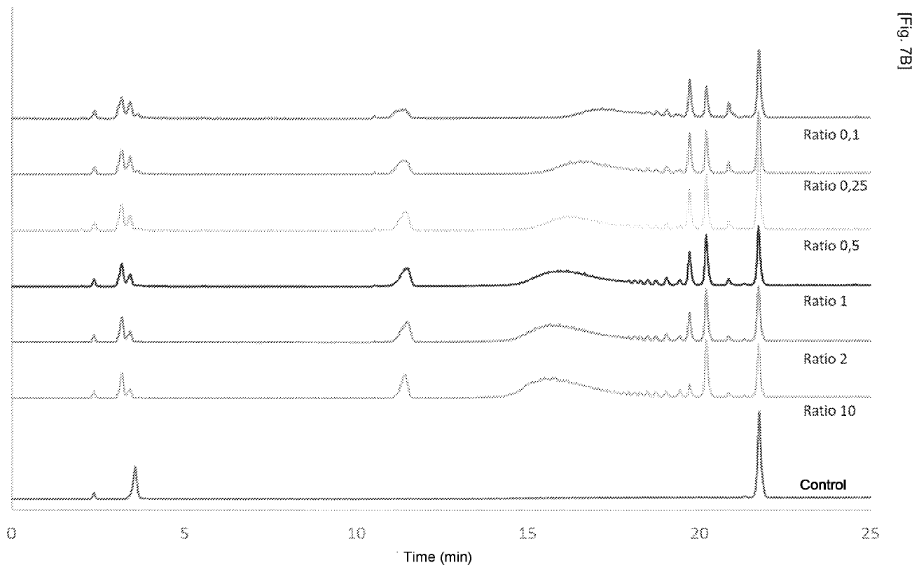

FIG. 5 shows the elongation profiles obtained with the enzyme DSR-M Δ1 (SEQ ID NO:1) with substrates of different geometries in terms of alkyl chain size (C8 to C16) and glucidic head (mono, diglucoside or oligoglucosides).

Glucansucrase enabled the elongation of all these acceptors.

The results also show the influence of the size of the acceptors. The longer the alkyl chain (i.e. the lower its solubility) the less efficient the glucosylation is under the tested reaction conditions.

This effect is counterbalanced by the increase in the size of the glucosidic head (glucosylation difference of C₁₂G₁ and C₁₂G₂). This shows the importance of controlling the synthesis conditions for an efficient elongation of the long alkyl chain APG.

### Example 5: Control of the Elongation Profile

5.1. Study of the Influence of the Alkyl Glucoside Acceptor:Sucrose Molar Ratio on the Elongation Profile

5.1.1 Materials and Methods

In a first series of experiments, the influence of the alkyl glucoside acceptor (i.e. alkyl glucoside of formula (II)):sucrose or sucrose analogue ratio was studied on the elongation profile of the alkyl glucoside acceptor C₈G₁ or C₁₂G₂ by the enzyme DSR-M Δ1 (SEQ ID NO: 1).

In this first series of experiments, the concentrations of C₈G₁ or C₁₂G₂ are fixed at 10 g/L (34.3 mM and 19.6 mM respectively) and the concentration of sucrose is modulated between 233.4 g/L (684 mM) and 2.3 g/L (6.8 mM) so that the alkyl glucosides acceptor:sucrose molar ratio is 0.05; 0.1; 0.25; 0.5; 1; 2; or 5.

Control: the control reactions represent the mixtures of C8G1 or C12G2 at a concentration of 10 g/L and 233.4 g/L of sucrose without the addition of enzyme

5.1.2. Results

The results of this first series of experiments are presented in FIG. 6 and confirm the possibility of modulating the elongation profile as a function of the alkyl glucoside acceptor:sucrose molar ratio between 0.05 and 5.

FIG. 6 shows the elongation profiles obtained with the enzyme DSR-M Δ1 (SEQ ID NO: 1) with decreasing molar ratios between C₈G₁ and sucrose (FIG. 6A) and between C₁₂G₂ and sucrose (FIG. 6B). The results show the effects of the alkyl glucoside acceptor:sucrose ratio on the elongation of the alkyl glucoside acceptor: the higher the molar ratio, the less the alkyl glucoside acceptor is extended. The results show that the average DP of the alkyl polyglucoside obtained is inversely proportional to the alkyl glucoside acceptor:sucrose molar ratio. Furthermore, FIG. 6A shows that for C₈G₁, the elongation profile varies significantly for molar ratios between 0.05 and 2, whereas molar ratios greater than 2 have little influence on the elongation profile.

FIG. 6B shows that for C₁₂G₂, the elongation profile varies significantly for molar ratios between 0.05 and 5.

5.2. Study of the Influence of the Glucansucrase Activity:Branching Sucrase Ratio on the Elongation Profile

5.2.1. Materials and Methods

In a second series of experiments, the influence of the glucansucrase activity:branching sucrase ratio was studied on the elongation profile of the alkyl glucoside acceptor C₈G₁.

In this second series of experiments, the glucansucrase is DSR-M Δ1 (SEQ ID NO: 1) and the branching sucrase BRS-A (SEQ ID NO: 18) or BRS-B Δ1 (SEQ ID NO: 19). The concentration of C₈G₁ is fixed at 10 g/L and the concentration of sucrose at 100 g/L. The quantities of DSR-M Δ1 and BRS-A or BSR-B are modulated so that the ratio DSR-M Δ1: BRS-A and DSR-M Δ1: BRS-B Δ1, expressed in enzyme activity units, is 0.1; 0.25; 0.5; 1; 2; 5 or 10, while respecting a total activity (DSR-M Δ1+BRS-A or BRS-B Δ1) of 1 U/mL. Control: the control reactions represent the mixture of C8G1 at a concentration of 10 g/L and 100 g/L of sucrose without the addition of enzyme.

5.2.2. Results

The results of the second series of experiments are presented in FIG. 7 and confirm the possibility of modulating the elongation profile as a function of the enzyme elongation activity:branching enzyme ratio.

FIG. 7 shows the elongation profiles obtained with the enzyme DSR-M Δ1 (SEQ ID NO: 1) on co-catalysis with the branching enzyme BRS-A (SEQ ID NO: 18) (FIG. 7A) or with the branching enzyme BRS-B Δ1 (SEQ ID NO: 19) (FIG. 7B). The results show the effect of the glucansucrase activity:branching sucrase ratio: the higher this ratio, the more extended the alkyl glucoside acceptor. The results show that the average DP of the alkyl polyglucoside obtained is proportional to the glucansucrase activity:branching sucrase ratio.

### REFERENCES

Bousquet, M.-P., Willemot, R.-M., Monsan, P. F., Paul, F. & Boures, E. Enzymatic Synthesis of α-Butylglucoside in a Biphasic Butanol-Water System Using the α-Transglucosidase from Aspergillus niger. in Carbohydrate Biotechnology Protocols (ed. Bucke, C.) 10, 291-296 (Humana Press, 1999).
Bousquet, M.-P., Willemot, R.-M., Monsan, P. & Boures, E. Production, purification, and characterization of thermostable α-transglucosidase from Talaromyces duponti-application to α-alkylglucoside synthesis. Enzyme and Microbial Technology 23, 83-90 (1998).
Dahiya, S., Ojha, S. & Mishra, S. Biotransformation of sucrose into hexyl-α-d-glucopyranoside and -polyglucosides by whole cells of Microbacterium paraoxydans. Biotechnol Lett 37, 1431-1437 (2015).
Ochs, M., Muzard, M., Plantier-Royon, R., Estrine, B. & Remond, C. Enzymatic synthesis of alkyl β-D-xylosides and oligoxylosides from xylans and from hydrothermally pretreated wheat bran. Green Chem. 13, 2380-2388 (2011).
Remond, Z. C., Ochs, M., Muzard, M., Plantier, R. R. & Estrine, B. Preparing surfactant compositions, comprises e.g. mixing lignocellulosic annual and perennial plant materials with water, contacting solution with strain or enzyme having xylanase activity to give composition of alkyl polypentosides. (2012).
Svensson, D., Ulvenlund, S. & Adlercreutz, P. Efficient synthesis of a long carbohydrate chain alkyl glycoside catalyzed by cyclodextrin glycosyltransferase (CGTase). Biotechnol. Bioeng. 104, 854-861 (2009).
Svensson, D. & Adlercreutz, P. Immobilisation of CGTase for continuous production of long-carbohydrate-chain alkyl glycosides: Control of product distribution by flow rate adjustment. Journal of Molecular Catalysis B: Enzymatic 69, 147-153 (2011).
Paul, C. J. et al. A GH57 4-α-glucanotransferase of hyperthermophilic origin with potential for alkyl glycoside production. Appl Microbiol Biotechnol 99, 7101-7113 (2015).
Vuillemin, M. et al. Characterization of the First α-(1->3) Branching Sucrases of the GH70 Family. Journal of Biological Chemistry 291, 7687-7702 (2016).
Côté, G. L. & Fobyt, J. F. Acceptor reactions of alternansucrase from Leuconostoc mesenteroides NRRL B-1355. Carbohydrate Research 111, 127-142 (1982).
Zhao, H. et al. Cyclomaltodextrin Glucanotransferase-Catalyzed Transglycosylation from Dextrin to Alkanol Maltosides. Bioscience, Biotechnology, and Biochemistry 72, 3006-3010 (2008).

1. A method for preparing an alkyl polyglucoside of formula (I)
[Glc]ₘ-[Glc]ₙ(-O—R) (I)
in which:
R represents a linear or branched, saturated or unsaturated alkyl group, comprising between 8 and 20 carbon atoms,
[Glc]ₘ-[Glc]ₙ represents a linear or branched glucoside part comprising n+m glucosyl units,
n+m being between 3 and 200;
said method comprising at least one step i) of elongating the glucoside chain of an alkyl glucoside of formula (II)
[Glc]ₙ(-O—R) (II)
in which:
R is as defined in formula (I),
[Glc]ₙ represents a glucoside part comprising n glucosyl units, n being between 1 and 15,
said step comprising placing said alkyl glucoside of formula (II) in contact with at least one α-transglucosylase of the GH70 family in the presence of sucrose or a sucrose analogue.

2. The method according to claim 1, wherein:
when R represents an alkyl group comprising between 12 and 20 carbon atoms, n+m is between 3 and 200, and
when R represents an alkyl group comprising between 8 and 12 carbon atoms, n+m is between 7 and 200.

3. The method according to claim 1, wherein step i) is carried out with an alkyl glucoside of formula (II) essentially consisting of molecules of alkyl monoglucoside, molecules of alkyl diglucoside, or a mixture thereof.

4. The method according to claim 1, wherein the α-transglucosylase of the GH70 family is a glucansucrase of the GH70 family, a branching sucrase of the GH70 family, or a mixture thereof.

5. The method according to claim 4, wherein the glucansucrase of the GH70 family has, as its amino acid sequence, a sequence chosen from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 and SEQ ID NO:15.

6. The method according to claim 4, wherein the branching sucrase of the GH70 family has, as its amino acid sequence, a sequence chosen from the group consisting of SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

7. The method according to claim 1, wherein step i) of glucoside elongation is carried out by simultaneously or successively placing the alkyl glucoside of formula (II) in contact with a plurality of α-transglucosylases of the GH70 family.

8. The method according to claim 1, wherein step i) comprises a step i₀) of placing the alkyl glucoside of formula (II) in contact with a mixture of one or more glucansucrases of the GH70 family and branching enzymes of the GH70 family.

9. The method according to claim 1, wherein step i) comprises a step ii) of placing the alkyl glucoside of formula (II) in contact with one or more glucansucrases of the GH70 family, then a step i²) of placing the alkyl polyglucoside obtained at the end of step ii) in contact with one or more branching sucrases of the GH70 family.

10. The method according to claim 1, wherein step i) is performed in solution at a pH that is controlled, in particular through the use of a buffer solution.

11. The method according to claim 10, wherein step i) is carried out at a pH value of between 5 and 8.

12. The method according to claim 1, wherein step i) is carried out with an initial concentration of sucrose or a sucrose analogue of between 20 and 660 g·L⁻¹.

13. The method according to claim 1, wherein step i) is preferably performed with a molar ratio of alkyl polyglucosides of formula (II): sucrose or sucrose analogue between 0.001 and 0.3, preferably between 0.002 and 0.006.

14. The method according to claim 1, wherein step i) is preferably implemented at a temperature of between 10° C. and 80° C.

15. The method according to claim 1, wherein step i) is carried out with α-transglucosylase of the GH70 family in solid form, in solution, in suspension, or immobilised.

16. The method according to claim 1, further comprising a step ii) of purifying the alkyl polyglucoside of formula (I) obtained at the end of step i).

17. An alkyl polyglucoside that can be obtained by implementing the method claim 1, characterised in that it is of formula (I):
[Glc]ₘ-[Glc]ₙ(-O—R) (I)
in which:
R represents a linear or branched, saturated or unsaturated alkyl group, comprising between 8 and 20 carbon atoms,
[Glc]ₘ-[Glc]ₙ represents a linear or branched glucoside part comprising n+m glucosyl units, n+m being between 3 and 200, preferably wherein, when R represents an alkyl group comprising 8 to 12 carbon atoms, n+m is between 7 and 200, and when R represents an alkyl group comprising 12 to 20 carbon atoms, n+m is between 3 and 200.

18. The alkyl polyglucoside according to claim 17, wherein the alkyl polyglucoside essentially comprises a mixture of alkyl polyglucoside molecules of formula (I), said mixture having an average degree of polymerisation between 3 and 25.

19. A method of using a polyglucoside of formula (I)
[Glc]ₘ-[Glc]ₙ(-O—R) (I)
in which:
R represents a linear or branched, saturated or unsaturated alkyl group, comprising between 8 and 20 carbon atoms,
[Glc]ₘ-[Glc]ₙ represents a linear or branched glucoside part comprising n+m glucosyl units,
n+m being between 3 and 200;
wherein said polyglucoside is preparing using a method comprising at least one step i) of elongating the glucoside chain of an alkyl glucoside of formula (II)
[Glc]ₙ(-O—R) (II)
in which:
R is as defined in formula (I),
[Glc]ₙ represents a glucoside part comprising n glucosyl units, n being between 1 and 15,
said step comprising placing said alkyl glucoside of formula (II) in contact with at least one α-transglycosylase of the GH70 family in the presence of sucrose or a sucrose analogue or as defined by claim 17 as a surfactant.

20. A method for glucoside elongation of an alkyl glucoside of formula (II) as defined in claim 1 comprising using an α-transglucosylase of the GH70 family.

21. The method according to claim 20, wherein said α-transglucosylase of the GH70 family is a glucansucrase of the GH70 family, a branching sucrase of the GH70 family, or a mixture thereof.


SEQUENCE LISTING (23 sequences)

SEQ ID NO: 1 - protein, 1391 aa, Leuconostoc citreum
  MISC_FEATURE (1)..(1): DSR-M Delta1
TPVGTTQSQQDLTGQTGQDKPTTKEVIDKKEPVPRVSAQNAGDLSADAKTTKADDKQDTQPTNAQLPDQGNKQTNSNSDKGVKESTTAPVKTTDVPSKSVAPETNTSINASDAISKSQEKQFEKAPDSVPETITGGRYSLKDGYYVYLDKQGKQVVGPKNIDNHLQYFDETTGKQVKGDFRSVNGKRIYFNANLGYADDYTTDVAGKLVGYDSNGNQVKAGYVTNSQGKTYYFNNQGEAIIGLKTDNNKTQYFGPDGAQVKGAFQQVNGKNIYFDAQTGYARQNVGFLDGTAKGFDEQGNQIKSGIATDLSGNVYYFDASGKMLTGVQNIDGKKYYFDEQGHRRRNYAGVFNNEFIYFGLDGVGQSAIEYQFEKGLTSQNSVATSHNAAKSYDTKSFTNVDGFLTANSWYRPTDILRNGTKWEPSTETDFRPLLMTWWPDKEVQANYLNYMSALGLGDQKIYTGASSQLDLNNAALIVQEAIEKKISLEKSTKWLDDSIKSFIKSKRKDIQGNLVDTNPGWTIDSETGSTNHLQNGAFIFTNSPLVPEANAAEGNRLINRTPSQQTGNHISYASQPYSGDDWGYELLLGNDVDNSNPIVQAEQLNWIHYLMNFGTITAPQDPDAHLANFDSIRIDAVDNVDADLLQIAGDYFKAAYQVGENDKNANQHIHILEDWSPNDVWYNQQVNGNSQLTMDATMQNQLLASLTRPITSRDSMKSFTKDALLVHRTADNSYNQAVPNYSFIRAHDSEVQTIIAKIISDKHPDLYPTVDKALLAKDSALYDEAFTEYNADMQKISSQKQYTHNNMPSAYAILLTNKDTVPRVYYGDLFTDNGEYMANKTPYYDAITSLLTARTKFVSGGQSLSVDKNDVLTSVRYGKGALSATDNGSSDTRNQGIGVIVSNNPNLDLNNDKVTLSMGISHAHQAYRPLLLTNSQGIVAYATDSEVPQNLYKTTNDKGELTFDASEIKGYDTVQTSGYLAVWVPVGASDEQDARTIASTEKNNGNSVYHSNAALDSQLIYEGFSNFQTVPSKNASADEYANVIIAKHAADFNKWGVTSFQMAPQYRSSTDGSFLDAVDTVQNGYAFTDRYDLGFNAADGSKNPTKYGTDEDLRNAIKSLHAQKTYDGSSIQVMADFVPDQLYNMPLEQAVSVIRTDKYGVNSENPDIQNIIYAANIKSSGTDYQSIYGGKYLAELQKNPLFKSLFDRIQISTKKTIDPNTRITQWSAKYFNGSNIQGKGINYVLKDWASNKYFNVSSNDDMYSRLPKQLMNQESNTGFIVDDIGVKYYSISGYQAKNTFVEDGNGEWYYFDNDGYMVKSTEESGPLRTVNASSKKYYILPNGVEIRNSFGQDIQGNTYYFDARGEMVTSQYISDDTQNIYYFNNDGTMAK

SEQ ID NO: 2 - protein, 894 aa, Leuconostoc citreum
  MISC_FEATURE (1)..(1): DSR-M Delta5
NSVATSHNAAKSYDTKSFTNVDGFLTANSWYRPTDILRNGTKWEPSTETDFRPLLMTWWPDKEVQANYLNYMSALGLGDQKIYTGASSQLDLNNAALIVQEAIEKKISLEKSTKWLDDSIKSFIKSKRKDIQGNLVDTNPGWTIDSETGSTNHLQNGAFIFTNSPLVPEANAAEGNRLINRTPSQQTGNHISYASQPYSGDDWGYELLLGNDVDNSNPIVQAEQLNWIHYLMNFGTITAPQDPDAHLANFDSIRIDAVDNVDADLLQIAGDYFKAAYQVGENDKNANQHIHILEDWSPNDVWYNQQVNGNSQLTMDATMQNQLLASLTRPITSRDSMKSFTKDALLVHRTADNSYNQAVPNYSFIRAHDSEVQTIIAKIISDKHPDLYPTVDKALLAKDSALYDEAFTEYNADMQKISSQKQYTHNNMPSAYAILLTNKDTVPRVYYGDLFTDNGEYMANKTPYYDAITSLLTARTKFVSGGQSLSVDKNDVLTSVRYGKGALSATDNGSSDTRNQGIGVIVSNNPNLDLNNDKVTLSMGISHAHQAYRPLLLTNSQGIVAYATDSEVPQNLYKTTNDKGELTFDASEIKGYDTVQTSGYLAVWVPVGASDEQDARTIASTEKNNGNSVYHSNAALDSQLIYEGFSNFQTVPSKNASADEYANVIIAKHAADFNKWGVTSFQMAPQYRSSTDGSFLDAVDTVQNGYAFTDRYDLGFNAADGSKNPTKYGTDEDLRNAIKSLHAQKTYDGSSIQVMADFVPDQLYNMPLEQAVSVIRTDKYGVNSENPDIQNIIYAANIKSSGTDYQSIYGGKYLAELQKNPLFKSLFDRIQISTKKTIDPNTRITQWSAKYFNGSNIQGKGINYVLKDWASNKYFNVSSNDDMYSRLPKQLMNQ

SEQ ID NO: 3 - protein, 894 aa, Leuconostoc citreum
  MISC_FEATURE (1)..(1): DSR-M Delta5 W624A
NSVATSHNAAKSYDTKSFTNVDGFLTANSWYRPTDILRNGTKWEPSTETDFRPLLMTWWPDKEVQANYLNYMSALGLGDQKIYTGASSQLDLNNAALIVQEAIEKKISLEKSTKWLDDSIKSFIKSKRKDIQGNLVDTNPGWTIDSETGSTNHLQNGAFIFTNSPLVPEANAAEGNRLINRTPSQQTGNHISYASQPYSGDDAGYELLLGNDVDNSNPIVQAEQLNWIHYLMNFGTITAPQDPDAHLANFDSIRIDAVDNVDADLLQIAGDYFKAAYQVGENDKNANQHIHILEDWSPNDVWYNQQVNGNSQLTMDATMQNQLLASLTRPITSRDSMKSFTKDALLVHRTADNSYNQAVPNYSFIRAHDSEVQTIIAKIISDKHPDLYPTVDKALLAKDSALYDEAFTEYNADMQKISSQKQYTHNNMPSAYAILLTNKDTVPRVYYGDLFTDNGEYMANKTPYYDAITSLLTARTKFVSGGQSLSVDKNDVLTSVRYGKGALSATDNGSSDTRNQGIGVIVSNNPNLDLNNDKVTLSMGISHAHQAYRPLLLTNSQGIVAYATDSEVPQNLYKTTNDKGELTFDASEIKGYDTVQTSGYLAVWVPVGASDEQDARTIASTEKNNGNSVYHSNAALDSQLIYEGFSNFQTVPSKNASADEYANVIIAKHAADFNKWGVTSFQMAPQYRSSTDGSFLDAVDTVQNGYAFTDRYDLGFNAADGSKNPTKYGTDEDLRNAIKSLHAQKTYDGSSIQVMADFVPDQLYNMPLEQAVSVIRTDKYGVNSENPDIQNIIYAANIKSSGTDYQSIYGGKYLAELQKNPLFKSLFDRIQISTKKTIDPNTRITQWSAKYFNGSNIQGKGINYVLKDWASNKYFNVSSNDDMYSRLPKQLMNQ

SEQ ID NO: 4 - protein, 1425 aa, Lactobacillus kunkeei
  MISC_FEATURE (1)..(1): GS-A
MNFSKNDRKIHFKMYKSGKQWITAGIMVGSVVFALTGINSNVSAKADTNQSVQQGSGSETKLESNVDAKSGSNSVSSKSGEVASTVVSSVANVSTESDSTVVSSAKPSSVTVAGANQQNSKAPSTSVESSTNNMDIASNASDEFKLHTKVIDGKTYYFNNNSELKSQVVVDNSTVYYFGNDGALTDVKSNFASGTIAASDKTAIYSADGNNITNVNGFITADSWYRPKQIQLNDNEWRDSTSNDFRPLLSVWWPTQKVEINYLNYMSQQGLVSGSFSSSNTSDQLNAAAATIRLNIEKNIQANNGSVDKIKTIFTNFVNSQSQWNIGSENYDMNDGFQGGALKYGNNSQTVDANSNYRLINRTPTQQDGNVNYKVSGYSGYEFLLGNDVDNSNPVVQAETLNWLHYLMNFGSITQNDKNANFDGVRVDAADNMDADILNIIGKYYRDAYGINKNDKNANDHIAILEDWSKNDAYYLKNHGADQMSIDGGYLGALQSSLMSNPSARTSISRFITYGLVNRRKDSTDNVNIPNYQIVRAHDNGVQDVIAKIIQDHIDPNSTGYTPSWSQINKAFKIYNDDQKRTNKKYTQYNIPSAYALILTNKDTTPRVYYGDMYTDNGQFMATKTPYYNSISEMLKGRVKYVAGGQAMKTVSVNKGKDKVLVSVRFGKGASSVTSKGTKHTRKSGIAVIESNNPKLKLGKKDKVVIYMGAAHKNQAYRPLVKTTKKGIATYSTDKSAKNSVIYTDSRGRLILTGSTIKGYANPQVSGYLSMWVPVGASAKQDVRTAPSSKTSKDGRSFHSNAALDSNVIFEAFSNFQSMPTKESQYSNVVIAKKAKLFSDLGFTNIELPPQYRSTTDGSFLDSTVQNGYAFNDRYDLGFNTPTKYGTAEQLATAIKALHAKKLKVLVDFVPDQLYTLPNKQVVSATRVNTVGKTSPNTNLINLLYEANSEGSGTDYQYKYGGEFLSDLQKMYPDLFTTKQISTGETIDPSKKIKIWDAQYLNGTNIQGRGAEYVLSNSYGSDYFTVKTLNNPNIDATILPKELLGQSVKYGIQMVNGKPQYITTSGYTAKNSFLMDESGNWYYVDENGDFVNSPRIINGYQYYFLPNGMNIRDSIVKESDGSLSYYQPNGTKAINSGYFVSDNIGQIIYITDTGKVYTGVLSSNGNDQFFDSNGYQVKNAMVMVNGKKEYFNNGSGNLVKNSFFSYNNNWYYANKKGYVVKGHQTINGKKYFFYNNGVQAKNTVIVNKNKTLSYYAKSNAQLVTKSFKYKGKKITIKKGAIYAPNKLVVIPAGKYLLDGKSHAKSGVHKVSGHYYSFDRSGKMKTSGTAKLKSVTYKVRSNGTLNFKTGKVVTAKDLKAVKKQLAAKKKAVKAETKKVSAAKKAVKRNSSKKNKATYKKSTKTLRELKASVKQFNSYIEKFNKRK

SEQ ID NO: 5 - protein, 1651 aa, Lactobacillus animalis
  MISC_FEATURE (1)..(1): GS-B
MFEKKLHYKMYKAGKHWVFAAIAVGIFGFASTTSALADETSSSNETQTEQTLNTDESTDTTTDVSNEAKATEAQLTTQDADMASSEEKTTNVEKEVTTAETNKDTTVKNVESSEQNTTTVADKNAVDSTAQVNTAEKENKYTQENVNGNWYLKDEQGNYLTGFQEIKDQNKTVYYNPDSKQMVYGQQNINGNWYLFDTFNGAMQTGLQYIRDQKKLAYYNEQGQMQYGTVEIDGQKYQADTFNGAIKGKGQTKIADNWYLFNNAGQVVDGWQWINDQGKTVYYSTKTAQMVHGQQNINGHWYLFDKTTGAMQRGFQNLKAYGDDKTVYYNQDGWMLYGQQKIDNKWYNFDTFNGAMKTGFVKIPEQNKTVYYAPNGQMQYGWQWVDNATRYFDTFNGAMATGQKLITGHWYLFDNNGAMQRGFQNLKNYGDNKTVYYNQDGWMLYGWQWVNNATRYFDTFNGAMTTGQKKINDHWYLFDKDGAMQRGIQYIPEENKLVYYNQDGWMLYGKQNINGVDHNFNTFNGALEAKGQVKVGNNWYLFNNSGTIQTGFQDLKAYGQDKVVYYDPKTAAMVYGYQNIDGNWYLFSRANGSMQRGLQNVNGVDLLFDEKTGALLTGVQNIKGNNYFVDKRSGNIKKNLVVLGADNKWMYFDAKTGKGTNTLEDQYKKGVVSGNVEFITNNAAYSFDGNSFENINGFLTADSWYRPKSILKDGSTWTATTETDLRPLLMTWWPNEQIKANYLNYMKDKGFINNSGTYNAESDPNYMDFAAQEAQRNIERKITKENDTTWLRDLITDFIKTQDIWNEQSEGVSTEGLQKFQGGFLKYVNSELTPYANSEWRKLGYQPTMLTQNNVGAEFLLANDIDNSNPVVQAEQLNWLHFLMNFGTITANDPSANFDGIRIDAVDNVDASLLSIAGDYFKAAYKVGQNDATANKHISILEDWNDKDPEYVNSIGNPQLTMDDYIVQQLKFSLGQAPDKVDRMQRFKEWYLVDRSKDNTENTAIPNYSFVRAHDASVQEDILQLIQDTTGKPWGVYTNEELQQGLKDYMADQKLTNKKYNRYNIPSSYAILLTNKDTIPRVYYGDLYSDAGKYMAEKSIYFDAIDNLLKTRTKYVAGGQTLDVDGHDILTSVRFGKGALNVTDKGTSETRTQGMGLIISNNNSLKLNDGEKVVLHMGAAHKNQAYRAVMLSSANGLINYTSDANAPVVYTNNDGDLIFTNKDVVTNGKVQANTAIKGVMNPYVSGYLAMWVPVGASATQDARTAASTKTTTDGSVFHSNAALDSNLIYEGFSNFQAFPENASENANAIIAQNVDLFNSWGVTSFQLAPQYVSSHDGSFLDSIIDNGYAFTDRYDLAMSKNNKYGSYQDLVNVLKALHAGGIQVIADWVPDQIYSLPGKEVVSVVRSDEFGNKVDGTQIDNTLYVVNTIGGGQYQKEYGGRYLEELKQKYPELFKTKQPSTGVTIDPSEKITEWSAKYLNGTNILHRGAEFVLRDGATYFRVAETSEVFLPSQLRGKITKNGFWKNDAGKVNYYNSEGEIMKNAFVKDGKNNWYYFDNDGNMVTNTALTIDSDAQVADYYFLNNGISLRDGFVQLANGDIYYYDVNGRKLKNGKVTVNNVEYTTDKNGKVVGENVLKKLDEIITTGKTTLI

SEQ ID NO: 6 - protein, 1703 aa, Lactobacillus apodemi
  MISC_FEATURE (1)..(1): GS-C
MFEKKLHYKMYKSGKHWVFAAITSIVFGAAMVNDVSADAVDTSSSDSQVEQVQTAETSTESSTSDSDSVANAETTTDSAATGNVTNESETQATATDQVASAQQQATTVENTDSTATTTENATTTATTTENAQADTTSNTEAATTTANTENSVTDDNAQTNNVSDTTATAATATTTSDKDESAQTTTQVNIDGNWYLQDASGNFVSGFQTITDQNKTVYYDPTTYQMQYGQQNIDGYWYLFDTFDGAMQTGLQYIKDQQKLVYYNADGQMQYGTVEIDGEQYHTDAITGAIIGEGQQKIAGHWYLYSTDNKVVNSWQYIADQGKTVYYDQNTAQMVYGQQNINGHWYLFDQTTGAMQRGFQNLKDYGEDKTVYYNQDGWMLYGQQKIDGYWYLFDTFDGAMQTGLQTLADQNKTVYYADNGQMQYGWQWVDNATRYFDTFDGAMVTGQQKIADHWYLFDQDGAMQRGFQNLKDYGEDKTVYYNQDGWMLYGWQWVENATRYFDTFNGAMATGQQKIAGYWYLFDQDGAMQRGFQFIPEQDKLVYYNQDGWMLYGEQNIDGVNYTFNTFDGALEATGQQKIAGHWYLFDENGNVLTGFQDLSAYGQDKVVYYDPTTAQILYGYQNIDGNWYLFDSYDGSLQKGIQTVNNVTLSFDTNTGALLTGVQNINGDLYYVDPISGTIKKNLMLLNADNTYTYFDATTGKGTNALTQQYEQGVESNNAEFVANNAAYSFDGDSFENVNGFLTADSWYRPKEILVNGTTWQASTESDFRPLLMTWWPDNQIRADYINFMKDNGLFDTDDTFTSDSDSSYMDYATEMIQRNIESKITTEGNTDWLRILMQEFVETEDIWNENSEDLRNTGMQKFQGGFLTYVNSDLTPWANSDYRKLGYQPAMISENSIGPEFLLANDVDNSNPVVQAEDLNWLHYLMNFGEITANDPSANFDGIRIDAVDNMDASILSLAGDYLRAHFNVGANDATANQHISILEDWDNRDATYTNSIGNPELTMDDYLVQQMKYSLGQSSETASSMKRFLEWYLVDRSVDNTENTAIPNYAFVRAHDASMQEDLMTLMQDKYGRSWGASFTVEEYEAAMEDYMEDQKATVKHYNRYNIPSSYAMLLTNKDVIPRVYYGDLFTDNGQYMETKSIYYDSIDNLLKTRIKYVSGGQTMDVDQNDILTSVRLGKDALNASDTGTAETRTTGIGLIISNNSNLTLADGDTVVLHMGAAHKNQAYRAAMIATDDGILNYASDDGAPIAYTDENGDLIFTNKDLVVNGQVQANTAIRGVLNPYVSGYVALWVPVGASETQDARTEASTEASTDGSVFHSNAALDSNVIYEGFSNFQAFPSDDSKNANALIAQNAELFKSWGVTSFQLAPQYVSTSDGTFIDSTIDNGYAFSDRYDLAMSKNNKYGSYQDLINAIKALHSVGIQVIADYVPDQVYSLSGQEVVTVVRTDEYGGKIDGTALDNVAYVVNTVGGGQYQAEYGGKFLEELQEKYPTLFTTKQVSTGETIDPSEKITQWSAKYFNGTNVLRRGVGYVLHDSNQYYRVAETDEVFLPSQLEGKVVDNGGEPFRTGFWTNEDGTVNYYDDMGKIMTNAFVKDGKNNWYYFDENGNMVSNTSLQITTDNTTAEYYFLANGVSVRNGFLQTASGDTYYYDENGRKLINGKVTVNSVEYTTDATGKVVEEKVLQKLDEIETAGRTSVF

SEQ ID NO: 7 - protein, 1116 aa, Lactobacillus animalis
  MISC_FEATURE (1)..(1): GS-D
GHWYLFNDQGEMQRGLQYLPKENKLVSYDHDGWMQYGEQTVGNKKYYFDTFNGALKAKGLNNISDHWFLFGNTGDVLTGEQEVNGEKLFFDKDTAQLKTGLQKIDSNIVYVDPKTASYKKNFLFNDNGKWYYFDSQTGIGSEKQLNSYMLTDLAVSDDYRNGNVLYSHDGENIENIDGYLTADSWYRPKEILDNGEHWRLSTTNDKRPLLMVWWPNKEVKVNYLNFMADKGLLQQNNFSTSDDSKELQRAAEEVQKNIEKKISSENGKTDWLRETLSSFIATQPMWNRESEDPAYFGFQFQGGFLKYVNNDLVPDTASDWRLMGRQPINVDGKGDQGAEFLLANDIDNSNPVVQAEQLNWLHYLLNFGNITANDSQANFDGIRIDAVDNVDADLLSIAGDYMRAAYGTSENDANANKHLSILEDWNINDAEYTKKIGTPQISIDNHTVTQITASLSKQQGGNDRMGRFLEWYMVDRSHDDSENVAIPSFAFVRAHDYAVQDMVQQATTDATGAEANHSNWDQLAKGLELYNADQAKTVKKYNRYNIPSSYALLLTNKDSIPRVYYGDVFTDGGQYMSQKSIYYDVIDNLLRSRVKYVAGGQTMAVDQHDILTSVRFGKGAMKVSDAGTDETRTEGIGVIISNNTDLKLSDNEQVVLHMGAAHRNQAYRPVVLSTNDGIENYTSDTDAPIMYTDNNGDLIFSNKDVVVNGNVLANTSVRGVTNPFVTGYLAVWVPVGAESDQDARTTASTEKHGSDEMFRSNAATDSNVIYEGFSNFQAPPASPEQAANVVIAKNVDLFKQWGITSFEFAPQYRSSTDGTFIDSIISNGYAFTDRYDLGFGEPTKYGTDSDLRQALRSLHEVGIQAIADWVPDQVYALPGKEVVAATRVDDHGNYIDGSQINDTLYVANTRGGGEYQSKFGGAFLEELKQKYPQLFQKAQISTGVPINGDVQIKEWSAKYMNGTNILSRGAGYVLKDENSGVYFNVDRNGFPTQLKNSSLDSVFVKNEDGSISYFKEGKLYRNQFLEKDGNWYYFDNDGHMVRENGESNNGFVILDDPEYSGVYFFMDNGVSFRNGFMTARDGATYYFDQYGRMIKNQKKEINGTEYNFDKDGRLIK

SEQ ID NO: 8 - protein, 1484 aa, Lactobacillus capillatus
  MISC_FEATURE (1)..(1): GS-E
MYKKGKNWVFATLAVTSISLGIAGTIDHVEAASPTVTDATTANQKSSETSKDDQSTAARQTVTADKTADKTADKTADKTAVTNNTVTDTTGQNNTQQTKSETKQSSSSQVAKNNTYTVPTKNNSSSSSSSSSSSSSSVKNTSNSSSVETARDQSDSSSAAQSVTSAKQEQPVDIDDTSINNQGGHYTNNSDTWTYRDQQGNLLKGLKKVDGNIQYFNPTTGAQIKGEYVSVKGKTYYFAKDSGNATAYTIDNNGTVKAYTADGTEVMTGFFTNQAGEKYYLTGSGQHAVGVKQINGTIYNFAQDGHLLKDVTETINGKLYYFDSQSGIGKELPKKEMQAPASVKAVGEYAAHNIAASYDKNSFENVNGYLNADSWYRPKYILQNGQSWVTTKPSDFRPLLMAWWPSKSFQANYLNYMKTKGLIKTDHQFTEKDSSLMLNDAVEQAQTAMEHAITVAGNTNWLRNIMSNFVKEEAVWNKSSEYVKYNGLQLQGGFLAYQNSKLTPWANSDYRQLGVTPGFLLGKSGKDAEFLLANDIDNSNPVVQAEQLNWLYYLTNFGSITANDDQANFDGIRVDAVDNVDADLLKIAADYFNAAYGVNKNDATANKHLSILEDWGLNDPWYVSQKGASQLTMDEYIAAQLNYSLTNTPGKNDKMARFIQWCLVNRSTDDTENTAIPNYSFVRAHDSNSQDQIRQAIKDATGGEYGKFTWADLEAGLQKFYADQNSTVKTYNKYNIPSAYAMLLTNKDTVPRIYYGDMYHEGGQYLSQKTIYYNTIANLLKTRIKYVAGGQTMAVDQHDILTSVRFGKGALNASAQGNAETRTQGIGVIVANNPTLKLGDGEKVILHMGAAHKNQEYRAVALTGSTGIENYTNDDAPIAFTDNNGDLIFTNQNLFINGKTELNTSIQGVANPEVSGYLAIWVPVGAAENQDARSEASTTSSTDGKVFHSNAALDSNVVFEGFSNFVAYPQVRTDNANVVIAKNADLFKSLGITSFELAPQYRSSGDGTFLDSTIDNGYAFTDRYDLGFNTPTKYGTVDDLRSAIKALHQAGIQAIADWVPDQLYSLSGQETVTVTRTDDHGNPYNDSGIKDILYVANTIGGGDYQKKYGGEFLALMQQKYPELFTEIQASTGKTIDAKTKLKSWSAKYLNGTNILGRGTGYVLKSNDNNQYFFVGSKGETFLPQVLVAEKDQQQYAAGDKSQSRFFIEKHDHNKWYLLDSITGKKQTGFQYLVDQGKTAYYNNAGQMQYGQQRIADSWYLFDKNTGAMQTGFQYIKDQHKTVYYNNAGQMQYNQQQVNGSWYLFDKNTGAMQTGFQRIKDQNKTVYYNNAGQMQYGQQRLNGHWYLFDSATGAQKTGFQYIKDQNKTAYYNNAGQMQYGQQRVNGHWYLFDKNTGAMQTGFQYIKDQHKTAYYNNAGQMQYGQQQVNGHWYLFDKNTGAMQTGFQYIRDQHKRVYYDAAGRMVYGRQKINNRWYNFDLRTGALK

SEQ ID NO: 9 - protein, 1708 aa, Leuconostoc fallax
  MISC_FEATURE (1)..(1): GS-F D1
ADKKAVTAEKQEVVTGGHYEAKGDGFVYISKDGKQLTGLQNIDGNVQYFGTDGLQVKGQFVEANGKTIYLDPNSGNAVANTQKVNGQLQGYDAQGNQVKKSFSNDASGNTYYFDDSGKMLTGLQSINGKSYYLDEQGHLKKNYAGIFNKQFMYFDAETGVGKTTIEYQFEQGLTSQNNENTPHNAAKSYDAKSFENVDGFLTADTWYRPTDILKDGQTWTASSDSDLRPLLMTWWPDKETQVSYLNYMQNVGLNDQPKVYTVDDSQNTLNEAAIGVQANIERKITLTNNTAWLRSEMEKFVNSRPAWNIDSENPDTSTTSLTKGFVKYLNNDATPDQNSKYRLFHRVLANLDGSNPSAGGFELLLANDVDNSNPVVQAEQLNWLYYLMNFGSITANDKDANFDSIRIDAVGNVDADLLKIAGDYFKQAYQIDQSDKNANQHISILENWDFDSVDKNAQLGNNQLTMDANQQFSLVDSLTNSANNRKSMLDYLNHGSLVSRKNTADNGTITPNYSFIRAHDSKVQENLASIISMMYPGTNGFMATQEQVDAAFKKYNEDEFGSNKVYTLSNLPSAYSLLLTNKDTIPRVYYGDLYTDNGKYMAEKTPYFEAIDALMKARIKYVAGGQSQHSGDEFVNGHDDILTSVRYGKGIDDANTVVDKNAEGVHEGIGVVISNNPNLNLANNEKVILNMGKSHANQDYRALLLTTKDGLKVYNTDTGAPVLRTNSDGQLILGNDLIYGVENVQVSGYLGAWVPVGAASDQDARTQSTDTVTNDGSSFHSNDSLDSQLIYEGFSNFQEKPTDSTPADEYTNAIIAKPESLELFKSWGVTSFQLAPQYRSSQDGTFIDSTIDNGYAFTDRYDLGFDTPTKYGNVQQLRDAIKALHSINTINNTGIQVMADWVPDQLYNFPDYEVVAVNKSTVNGDAVNDSELNDVLYAAKTKGGGKYQAQYGGEYLSLLQSLYPTLFSEIQATTGEPIDPSVKIKEWSAKYLNGSSLQGRGAHYVLKDWASNQYFNIAKTDEVFLPLQLMNKESNTGFTSDATGVKYYSTSGYQAKDTFIEDGNGNWYYFDDKGYMARSQDGKSPLTVISTNTDTKNGTYYFMPNGVELRDGFGQDDSGNTYYFDKLGKMVTNQYIFDKANNTYHVNSDGTMSRGLVWIDNNLQYFAANGVQLKGVYAKDTDNKLYYFDANTGNNDSDFANKSWQSNWTTNDYIAIERDAVNTIGVRTDYTAYISSAIRQDGIYKDAPLGTSVKDQNNQTVSASLVGNTSEYNHQKVQVSRQYTDSQGVVWNLINFENKTWWVDSNALVTVNFTSQKPTKHFVQFGMRQGKYDGFYLSAPYKQTESKWVASTRTHQGQLLEVVGQYTTGSGSRKVTWYLVGLDGKQVWVDSRAVGTNFSHKTNINLLINSATRNDGMYLNAPYGQKGYKRETSSRFYNEKLVTVSQQYYDNKGVIWNLITLNGKQLWVDSRAFASTIEKSINQKMFIDSSQRNDGMYLNAPYRAQGAKRSASTKTYNGQLVQVTKQRKDSHGVTWYLATVNNQTLWVDSRAFATVIDKKVNQNLYINSRNDGMYLNAPYRAQGAKRYASTKTYTGQRVQVTLQRKDTHGVTWYLTKVDGKQLWVDSHAFAPTFTRNVSLNVKVNSSKRNDGIYLNAPYGNKKAKRIASTKAYNGKRVKASKEYKDAKGVTWYLVNLNNKQVWIDKRAF

SEQ ID NO: 10 - protein, 1579 aa, Streptococcus salivarius
  MISC_FEATURE (1)..(1): GS-FS
MEQKVRFKMHKVKKNWVTIGVTALSMVTVAGGTLLVDQQVQADEQNPANQSGDSSQNLLREAPATTNDAATTVAPTISADANTASVSIPVADATSTTTDVTDRAAAPAANTATVDTNSGQAAPSTNVQAAAADTSATTTDTNNATATATTDRAATTDTANTEARTRSRRALAETREANTNTATGIQWINGKQYYVNSDGSVRKNFVFEQDGKSYYFDAETGALATKSQDEFSTKPIKATVDFSSGNQLYKNDNKSLDQLDSFITADAWYRPKSILKDGKTWTASTEADKRPLLMVWWPDKSTQVNYLNYMQNQGLGAGSFSTNSSQESLNLAAKTVQTKIEERIAREGNTNWLRTSIDQFIKTQPGWNSSTENSSYDHLQGGQLLFNNSKGDTGNRTSYANSDYRLLNRTPTNQTGTRKYFKDNSIGGLEFLLANDIDNSNPAVQAEQLNWLHFMMNIGSIMANDPTANFDGLRVDALDNVDADLLQIASDYFKAAYGVDKSEANAIKHLSYLEAWSANDPYYNKDNKGAQLPIDNALRNALTNLLMRDKNTRMQLGDMTAFMNSSLNPRGANDKNGERMANYIFTRAHDTEAQTIIQRIIRDRINPNLFGYNFTRDEIKKAFEIYNADIDKAHKTYASYNLPSVYALMLTNKDSVTRVYYGDLYREDGHYMAKKTPYFDAIDTLLRARIKYVAGGQDMEVKKVGNDGLLTSVRYGKGANNRTDWGTAETRTQGMGVIMTNNYDFRLGSNETVTMNMGRAHRNQLYRPLLLTTKDGIATYLNDSDVPKNLLKRTDWNGNLTFNANDVFGVENVQVSGYLGVWVPVGAKANQDARTQPSNRANSDGQVYKSSAALDSQVMYEAFSNFQAFADNQPELYMNRVLAKNTNLLKAWGVTSVGLPPQYVSSKDGTFLDSTIDNGYAFDDRYDMALSQNNKYGSLEDLLNVLRALHKDGIQAIADWVPDQIYNLPGKEVVNATRVNGYGNHQGGYQIIDQAYVANTRTDGTDYQGKYGGAFLDELKAKYPSIFNRVQISNGKQLPTNEKITKWSAKYFNGTNILGRGINYVLRDDKTNQYFNTSANGQLLPTPLRDTGAITSTQVFQRRGQDVYFLRDNQTVKNEFVQDGSGNWYYFGGDGKMTKGARNINNKDYYFFDNGIQLRNALRRASNGYTYYYGLDGAMVKNAFVDFDDKHKQVRAFTTQGTMVVGNLHWSSHHFYFDRETGIQAKDRVVRTDDGKLHYYVAETGDMGRNVFATDSSTGKRYYFDADGNTVTGSRVIDGKTYYFNQDGSVGTAYSNRADSIIFENGKARYITPAGEIGRSIFVYNPATKAWNYFDKEGNRVTGRQYIDGHLYYFKEDGSQAKGEIIEENGIKYYYEPESGILASGRYLQVGDDQWMYFKQDGSLAIGQVRADHGYLKYFDKNGIQVKGKTIVEDGKTYYYDAHSGALVTSSFAEIAPNQWSYFNTDGQALKGKWTINGKEYYFDQNGIQYKGKAVKVGSRYKYYDENDGQPVTNRFAQIEPNVWAYFGADGYAVTGEQVINGQHLYFDQSGRQVKGAYVTVNGQRRYYDANTGEYVPGR

SEQ ID NO: 11 - protein, 2621 aa, Lactobacillus kunkeei
  MISC_FEATURE (1)..(1): DSR-G
MQSYESVSLSVSLASNASQNLKSHTKNIDGKTYFYDLNNNEVKSALIDDNNTYYYFGPNGYLTTFDDSKFSDGSINSSDQLSIYTPDGKSITNVDGFLTADSWYRPKQIQVSDTQWRNSTNADFRPLLSVWWPNKTVEINYLNYMSKNGLVNGHFDNNSSANDINQAAATVRLSIENKIKADNGDLSAIRSLFTTFINSQDEWNIDSEDYNSGDGLQGGSLLFGNNSETKDANSNYRLLNRNPTQQDGKIDYTHTQDPGFEFLLANDVDNSNPVVQAETLNWLHYLMNFGSIVNKDSSANFDGVRVDAVDNMDADVLNIISQYFRDAYKINANDVNSNNHLSILEDWSGNDPYYQKDHGTNQMTLDSDYLSSLRSILMYNPSVRSDMSTIINAGIVDRADDNTTNKAIPNYEIVRAHDAGVQDIISQIIIDNIDPHASASNPTWEQMREAFKIYDADENSTVKKYTQYNIPASYALTLTNKDTTPRVYYGDLYTDDGQFMEQKTPYYDAIAEMLKSRVKYVAGGQHMEAVKVNNGEDTILTSVRYGKDALNADDLGDSLTRTSGIAVVESNNPTLSLSDKDKVVIHMGAAHKNQEYRQLLTASNSGIDSFDSDSSKGYVVVRTDDNGDLTLDGNIIKGYANPQVSGYLSMWVPLGASDNQDIRTAPSSESSTDGQSIHSNEASDSNVILEAFSNFQNFPQTTDQYENVVIQKNAEDFKNLGFTYLELPPQYKSTKDGSFIDSVVQNGYSFNDRYDLGFDTPTKYGTAEQLIDAIKSLHAQGIKVLADIVPDQIYSLPNQQIVNATRTNPYGDANFNSNLINVLYDAFSKGSGTDYQYKYGGEFLEQLKKLYPDLFTTKQISTGQPIDASKKLKIWTAEYLNGSNIQGRGSGYVLSSTPNSNYYTVVDDGNTNIKSSNLPKGLLGDNVEYGLKLIDGKMKYVSTGGFIAKNTFIQDDNHNWYYVDNNGDFVTSPQVINGNKYFFLSNGVNLRDFISVNSDGTMNYYQSNGILANNPGYYYSNNINQMVHVNNNGVLDTGIVNVNGYVQYFDDNGYQVKGDIVNFNGRKMYFDDGSGNLVYNRFVSYNGNWYYAGSNGLLVNGLQNINNQSLYFDDNGKQVKGGMISLDNHKMYFDANSGNLYKNSFVLLDGDVYYANNDGYIVNGYQNIGGRNLYFDSDGKQVKDQFVNINGNKVYFNGTDGSEVKDDFIIHDDKEYYADNQGHLLTGYNFVNGQNMYFNEDGSQVKNSIVSLDGKLMYFDENSGNQVKNGFILHNGNVYYSNKDGILVTGYQNIDGQDLFFNADGTQIKGGTAEIDGVNYYFENGEGHLVGKVDQVINSNRFSDNKLLDANNNVVKGLIFNNGQLQYFDSLTGDQAKNKQIIADGNTYYFDNSGNGTYLFTNIGESETNDFSQRNAANSVNLSDYKNVVDGFFTADTWYRPKQILDNGTTWRNSNSNDFRPMITVWWPNKNVQVNYLKLMQNNNLLDKSTNYTLQSNQQMLNQAAQNAQVNIEKKIAQTGNTDWLNDLLFKGNGDTPSFVKQQYIWNILSESPGQDDALLQNGYFKYVNSELTPNTNSSYRISNNLCDFLLANDVDNSNPAVQAEDLNWLYYLTNFGTITANDPNANFDSIRIDAYAFINNDIVQRSNDYMKQKYKLTESSNNANSHLSIVEAGVDAGTTSTNNDELVESPFRTISYGLIHKDRNPQDMNNFIKEVDTGVVIADHENNSQEIGQPNYSIVHAHDKDIQDKVGEAIVDATGIKDWTNFTPSELSAGLKLYYDDQRSSEKKYNDYNIPSAYAIMLTNKGTVPRIYYGDMYQDDGQFMQKQSVYYDDIVSLLTARKKYVSGGQSMSVDHNNFLESVRFGKGAGSESDSGNAETRNEGIGLIVGNDQNKKLNDGDTVVLHMGAAHRNQKYRALMLTTNDGIKNYDSDENAPIAETDSNGDLVFSNKDINGQANTSVRGVLNPEVAGYVAAWVPLGASDDQDSRTLSSNKSYNDGKVLHSGDDLDSNVIFEAFSNFQPEPTNENEYENVVIPQKASLFKDWGITSFELPPQYRSSNDHTFVDATINNGYAFSDRYDLGFGQPTKYGTDVDLRNTIKSLHDNGMQVMADVVYNQLYNLPGQEVVSAVRAGFTGNTVSLPFGNQLYVVNTVGGGDYQKKYGGAFLQKLYQEYPSLFDSEKYQYNSKNYVTDLLVMTDGERSAIPSDQPITEWSAKYMNGTNILGRGMGYVLKDWNTGTYFKLNGNNSVLPDVLTYRNSFDDNTGTQSSSSAQSSASNHETQISSSAQSSASNHEAQSSSSAQSSVSSHEVQSSSSAQSSASSHEVQSSSSAQSSASSHEVQSSSSAQSSASSNEAQSSSSAQSSASSHEAQKSSSAQSSASSHGEQGKTSAHGNGNGVSQNKPSQSHANDDSDKVLSNRKLTTNNQVSNDHSVNNQTTQHVSNVNTLSRTKTRENQKIQSKMLTTKSLKQVNKQASSQEKLVKKELKQLDKLKAELKKHSSKKLKKEYNALLQKYKANKKYYLALVAEQNTVEKYLKSAKNLKKDKKALNSLNKKLAKDKKELKRHTSKKLKKTYEKDIKGKKKLTKAIKKYKGDEAKLKKAIVNNFKKASSMKSKKK

SEQ ID NO: 12 - protein, 1407 aa, Lactobacillus kunkeei
  MISC_FEATURE (1)..(1): DSR-G CD1
MQSYESVSLSVSLASNASQNLKSHTKNIDGKTYFYDLNNNEVKSALIDDNNTYYYFGPNGYLTTFDDSKFSDGSINSSDQLSIYTPDGKSITNVDGFLTADSWYRPKQIQVSDTQWRNSTNADFRPLLSVWWPNKTVEINYLNYMSKNGLVNGHFDNNSSANDINQAAATVRLSIENKIKADNGDLSAIRSLFTTFINSQDEWNIDSEDYNSGDGLQGGSLLFGNNSETKDANSNYRLLNRNPTQQDGKIDYTHTQDPGFEFLLANDVDNSNPVVQAETLNWLHYLMNFGSIVNKDSSANFDGVRVDAVDNMDADVLNIISQYFRDAYKINANDVNSNNHLSILEDWSGNDPYYQKDHGTNQMTLDSDYLSSLRSILMYNPSVRSDMSTIINAGIVDRADDNTTNKAIPNYEIVRAHDAGVQDIISQIIIDNIDPHASASNPTWEQMREAFKIYDADENSTVKKYTQYNIPASYALTLTNKDTTPRVYYGDLYTDDGQFMEQKTPYYDAIAEMLKSRVKYVAGGQHMEAVKVNNGEDTILTSVRYGKDALNADDLGDSLTRTSGIAVVESNNPTLSLSDKDKVVIHMGAAHKNQEYRQLLTASNSGIDSFDSDSSKGYVVVRTDDNGDLTLDGNIIKGYANPQVSGYLSMWVPLGASDNQDIRTAPSSESSTDGQSIHSNEASDSNVILEAFSNFQNFPQTTDQYENVVIQKNAEDFKNLGFTYLELPPQYKSTKDGSFIDSVVQNGYSFNDRYDLGFDTPTKYGTAEQLIDAIKSLHAQGIKVLADIVPDQIYSLPNQQIVNATRTNPYGDANFNSNLINVLYDAFSKGSGTDYQYKYGGEFLEQLKKLYPDLFTTKQISTGQPIDASKKLKIWTAEYLNGSNIQGRGSGYVLSSTPNSNYYTVVDDGNTNIKSSNLPKGLLGDNVEYGLKLIDGKMKYVSTGGFIAKNTFIQDDNHNWYYVDNNGDFVTSPQVINGNKYFFLSNGVNLRDFISVNSDGTMNYYQSNGILANNPGYYYSNNINQMVHVNNNGVLDTGIVNVNGYVQYFDDNGYQVKGDIVNFNGRKMYFDDGSGNLVYNRFVSYNGNWYYAGSNGLLVNGLQNINNQSLYFDDNGKQVKGGMISLDNHKMYFDANSGNLYKNSFVLLDGDVYYANNDGYIVNGYQNIGGRNLYFDSDGKQVKDQFVNINGNKVYFNGTDGSEVKDDFIIHDDKEYYADNQGHLLTGYNFVNGQNMYFNEDGSQVKNSIVSLDGKLMYFDENSGNQVKNGFILHNGNVYYSNKDGILVTGYQNIDGQDLFFNADGTQIKGGTAEIDGVNYYFENGEGHLVGKVDQVINSNRFSDNKLLDANNNVVKGLIFNNGQLQYFDSLTGDQAKNKQIIADGNTYYFDNSGNGTYLFTNIGESE

SEQ ID NO: 13 - protein, 1425 aa, Leuconostoc mesenteroides
  MISC_FEATURE (1)..(1): ASR D1
MKQQETVTRKKLYKSGKVWVAAATAFAVLGVSTVTTVHADTNSNVAVKQINNTGTNDSGEKKVPVPSTNNDSLKQGTDGFWYDSDGNRVDQKTNQILLTAEQLKKNNEKNLSVISDDTSKKDDENISKQTKIANQQTVDTAKGLTTSNLSDPITGGHYENHNGYFVYIDASGKQVTGLQNIDGNLQYFDDNGYQVKGSFRDVNGKHIYFDSVTGKASSNVDIVNGKAQGYDAQGNQLKKSYVADSSGQTYYFDGNGQPLIGLQTIDGNLQYFNQQGVQIKGGFQDVNNKRIYFAPNTGNAVANTEIINGKLQGRDANGNQVKNAFSKDVAGNTFYFDANGVMLTGLQTISGKTYYLDEQGHLRKNYAGTFNNQFMYFDADTGAGKTAIEYQFDQGLVSQSNENTPHNAAKSYDKSSFENVDGYLTADTWYRPTDILKNGDTWTASTETDMRPLLMTWWPDKQTQANYLNFMSSKGLGITTTYTAATSQKTLNDAAFVIQTAIEQQISLKKSTEWLRDAIDSFVKTQANWNKQTEDEAFDGLQWLQGGFLAYQDDSHRTPNTDSGNNRKLGRQPINIDGSKDTTDGKGSEFLLANDIDNSNPIVQAEQLNWLHYLMNFGSITGNNDNANFDGIRVDAVDNVDADLLKIAGDYFKALYGTDKSDANANKHLSILEDWNGKDPQYVNQQGNAQLTMDYTVTSQFGNSLTHGANNRSNMWYFLDTGYYLNGDLNKKIVDKNRPNSGTLVNRIANSGDTKVIPNYSFVRAHDYDAQDPIRKAMIDHGIIKNMQDTFTFDQLAQGMEFYYKDQENPSGFKKYNDYNLPSAYAMLLTNKDTVPRVYYGDMYLEGGQYMEKGTIYNPVISALLKARIKYVSGGQTMATDSSGKDLKDGETDLLTSVRFGKGIMTSDQTTTQDNSQDYKNQGIGVIVGNNPDLKLNNDKTITLHMGKAHKNQLYRALVLSNDSGIDVYDSDDKAPTLRTNDNGDLIFHKTNTFVKQDGTIINYEMKGSLNALISGYLGVWVPVGASDSQDARTVATESSSSNDGSVFHSNAALDSNVIYEGFSNFQAMPTSPEQSTNVVIATKANLFKELGITSFELAPQYRSSGDTNYGGMSFLDSFLNNGYAFTDRYDLGFNKADGNPNPTKYGTDQDLRNAIEALHKNGMQAIADWVPDQIYALPGKEVVTATRVDERGNQLKDTDFVNLLYVANTKSSGVDYQAKYGGEFLDKLREEYPSLFKQNQVSTGQPIDASTKIKQWSAKYMNGTNILHRGAYYVLKDWATNQYFNIAKTNEVFLPLQLQNKDAQTGFISDASGVKYYSISGYQAKDTFIEDGNGNWYYFDKDGYMVRSQQGENPIRTVETSVNTRNGNYYFMPNGVELRKGFGTDNSGNVYYFDDQGKMVRDKYINDDANNFYHLNVDGTMSRG

SEQ ID NO: 14 - protein, 1455 aa, Streptococcus mutans
  MISC_FEATURE (1)..(1): GTF-SI
MEKKVRFKLRKVKKRWVTVSVASAVVTLTSLSGSLVKADSTDDRQQAVTESQASLVTTSEAAKETLTATDTSTATSATSQPTATVTDNVSTTNQSTNTTANTANFDVKPTTTSEQSKTDNSDKIIATSKAVNRLTATGKFVPANNNTAHSRTVTDKIVPIKPKIGKLKQPSSLSQDDIAALGNVKNIRKVNGKYYYYKEDGTLQKNYALNINGKTFFFDETGALSNNTLPSKKGNITNNDNTNSFAQYNQVYSTDAANFEHVDHYLTAESWYRPKYILKDGKTWTQSTEKDFRPLLMTWWPDQETQRQYVNYMNAQLGIHQTYNTATSPLQLNLAAQTIQTKIEEKITAEKNTNWLRQTISAFVKTQSAWNSDSEKPFDDHLQKGALLYSNNSKLTSQANSNYRILNRTPTNQTGKKDPRYTADNTIGGYEFLLANDVDNSNPVVQAEQLNWLHFLMNFGNIYANDPDANFDSIRVDAVDNVDADLLQIAGDYLKAAKGIHKNDKAANDHLSILEAWSDNDTPYLHDDGDNMINMDNKLRLSLLFSLAKPLNQRSGMNPLITNSLVNRTDDNAETAAVPSYSFIRAHDSEVQDLIRDIIKAEINPNVVGYSFTMEEIKKAFEIYNKDLLATEKKYTHYNTALSYALLLTNKSSVPRVYYGDMFTDDGQYMAHKTINYEAIETLLKARIKYVSGGQAMRNQQVGNSEIITSVRYGKGALKATDTGDRTTRTSGVAVIEGNNPSLRLKASDRVVVNMGAAHKNQAYRPLLLTTDNGIKAYHSDQEAAGLVRYTNDRGELIFTAADIKGYANPQVSGYLGVWVPVGAAADQDVRVAASTAPSTDGKSVHQNAALDSRVMFEGFSNFQAFATKKEEYTNVVIAKNVDKFAEWGVTDFEMAPQYVSSTDGSFLDSVIQNGYAFTDRYDLGISKPNKYGTADDLVKAIKALHSKGIKVMADWVPDQMYALPEKEVVTATRVDKYGTPVAGSQIKNTLYVVDGKSSGKDQQAKYGGAFLEELQAKYPELFARKQISTGVPMDPSVKIKQWSAKYFNGTNILGRGAGYVLKDQATNTYFSLVSDNTFLPKSLVNPNHGTSSSVTGLVFDGKGYVYYSTSGNQAKNAFISLGNNWYYFDNNGYMVTGAQSINGANYYFLSNGIQLRNAIYDNGNKVLSYYGNDGRRYENGYYLFGQQWRYFQNGIMAVGLTRIHGAVQYFDASGFQAKGQFITTADGKLRYFDRDSGNQISNRFVRNSKGEWFLFDHNGVAVTGTVTFNGQRLYFKPNGVQAKGEFIRDADGHLRYYDPNSGNEVRNRFVRNSKGEWFLFDHNGIAVTGTRVVNGQRLYFKSNGVQAKGELITERKGRIKYYDPNSGNEVRNRYVRTSSGNWYYFGNDGYALIGWHVVEGRRVYFDENGVYRYASHDQRNHWDYDYRRDFGRGSSSAVRFRHSRNGFFDNFFRF

SEQ ID NO: 15 - protein, 1518 aa, Streptococcus salivarius
  MISC_FEATURE (1)..(1): GTF-J
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 16 - protein, 1068 aa, Leuconostoc citreum
  MISC_FEATURE (1)..(1): GBD-CD2
QAGHYITKNGNDWQYDTNGELAKGLRQDSNGKLRYFDLTTGIQAKGQFVTIGQETYYFSKDHGDAQLLPMVTEGHYGTITLKQGQDTKTAWVYRDQNNTILKGLQNINGTLQFFDPYTGEQLKGGVAKYDDKLFYFESGKGNLVSTVAGDYQDGHYISQDGQTRYADKQNQLVKGLVTVNGALQYFDNATGNQIKNQQVIVDGKTYYFDDKGNGEYLFTNTLDMSTNAFSTKNVAFNHDSSSFDHTVDGFLTADTWYRPKSILANGTTWRDSTDKDMRPLITVWWPNKNVQVNYLNFMKANGLLTTAAQYTLHSDQYDLNQAAQDVQVAIERRIASEHGTDWLQKLLFESQNNNPSFVKQQFIWNKDSEYHGGGDAWFQGGYLKYGNNPLTPTTNSDYRQPGNAFDFLLANDVDNSNPVVQAENLNWLHYLMNFGTITAGQDDANFDSIRIDAVDFIHNDTIQRTYDYLRDAYQVQQSEAKANQHISLVEAGLDAGTSTIHNDALIESNLREAATLSLTNEPGKNKPLTNMLQDVDGGTLITDHTQNSTENQATPNYSIIHAHDKGVQEKVGAAITDATGADWTNFTDEQLKAGLELFYKDQRATNKKYNSYNIPSIYALMLTNKDTVPRMYYGDMYQDDGQYMANKSIYYDALVSLMTARKSYVSGGQTMSVDNHGLLKSVRFGKDAMTANDLGTSATRTEGLGVIIGNDPKLQLNDSDKVTLDMGAAHKNQKYRAVILTTRDGLATFNSDQAPTAWTNDQGTLTFSNQEINGQDNTQIRGVANPQVSGYLAVWVPVGASDNQDARTAATTTENHDGKVLHSNAALDSNLIYEGFSNFQPKATTHDELTNVVIAKNADVFNNWGITSFEMAPQYRSSGDHTFLDSTIDNGYAFTDRYDLGFNTPTKYGTDGDLRATIQALHHANMQVMADVVDNQVYNLPGKEVVSATRAGVYGNDDATGFGTQLYVTNSVGGGQYQEKYAGQYLEALKAKYPDLFEGKAYDYWYKNYANDGSNPYYTLSHGDRESIPADVAIKQWSAKYMNGTNVLGNGMGYVLKDWHNGQYFKLD

SEQ ID NO: 17 - protein, 1877 aa, Leuconostoc citreum
  MISC_FEATURE (1)..(1): BRS-A
MRQKETITRKKLYKSGKSWVAAATAFAVMGVSAVTTVSADTQTPVGTTQSQQDLTGQRGQDKPTTKEVIDKKEPVPQVSAQNAGDLSADAKTTKADDKQDTQPTNAQLPDQGNKQTNSNSDKGVKESTTAPVKTTDVPSKSVTPETNTSINGGQYVEKDGQFVYIDQSGKQVSGLQNIEGHTQYFDPKTGYQTKGELKNIDDNAYYFDKNSGNGRTFTKISNGSYSEKDGMWQYVDSHDKQPVKGLYDVEGNLQYFDLSTGNQAKHQIRSVDGVTYYFDADSGNATAFKAVTNGRYAEQTTKDKDGNETSYWAYLDNQGNAIKGLNDVNGEIQYFDEHTGEQLKGHTATLDGTTYYFEGNKGNLVSVVNTAPTGQYKINGDNVYYLDNNNEAIKGLYGINGNLNYFDLATGIQLKGQAKNIDGIGYYFDKDTGNGSYQYTLMAPSNKNDYTQHNVVNNLSESNFKNLVDGFLTAETWYRPAQILSHGTDWVASTDKDFRPLITVWWPNKDIQVNYLRLMQNEGVLNQSAVYDLNTDQLLLNEAAQQAQIGIEKKISQTGNTDWLNNVLFTTHDGQPSFIKQQYLWNSDSEYHTGPFQGGYLKYQNSDLTPNVNSKYRNADNSLDFLLANDVDNSNPIVQAEDLNWLYYLLNFGSITTQGKENNSNFDSIRIDAVDFVSNDLIQRTYDYLRAAYGVDKNDKEANAHLSLVEAGLDAGTTTIHQDALIESDIREAMKKSLTNGPGSNISLSNLIQDKEGDKLIADRANNSTENVAIPNYSIIHAHDKDIQDKVGAAITDATGADWTNFTPEQLQKGLSLYYEDQRKIEKKYNQYNIPSAYALLLTNKDTVPRVYYGDMYQDDGQYMQKQSLYFDTITALMEARKQFVAGGQTINVDDNGVLTSVRFGKGAMTANDIGTNETRTQGIGVVIANDPSLKLSKDSKVTLHMGAAHRNQNYRALLLTTDNGIDSYSSSKNAPVIKTDDNGDLVFSNQDINDQLNTKVHGFLNSEVSGYLSAWVPLDATEQQDARTLPSEKSVNDGKVLHSNAALDSNLIYEAFSNFQPMPTNRNEYTNVVIADKADTFKSWGITSFEMAPQYRSSQDKTFLDSTIDNGYAFTDRYDLGFEKPTKYGNDEDLRQAIKQLHSSGMQVMADVVANQIYNLPGKEVASTNRVDWNGNNLSTPFGTQMYVVNTVGGGKYQNKYGGEFLDKLKAAYPDIFRSKNYEYDVKNYGGNGTGSVYYTVDSKTRAELDTDTKIKEWSAKYMNGTNVLGLGMGYVLKDWQTGQYFNVSNQNMKFLLPSDLISNDITVQLGVPVTDKKIIFDPASAYNMYSNLPEDMQVMDYQDDKKSTPSIKPLSSYNNKQVQVTRQYTDSKGVSWNLITFAGGDLQGQKLWVDSRALTMTPFKTMNQISFISYANRNDGLFLNAPYQVKGYQLAGMSNQYKGQQVTIAGVANVSGKDWSLISFNGTQYWIDSQALNTNFTHDMNQKVFVNTTSNLDGLFLNAPYRQPGYKLAGLAKNYNNQTVTVSQQYFDDQGTVWSQVVLGGQTVWVDNHALAQMQVRDTNQQLYVNSNGRNDGLFLNAPYRGQGSQLIGMTADYNGQHVQVTKQGQDAYGAQWRLITLNNQQVWVDSRALSTTIMQAMNDDMYVNSSQRTDGLWLNAPYTMSGAKWAGDTRSANGRYVHISKAYSNEVGNTYYLTNLNGQSTWIDKRAFTATFDQVVALNATIVARQRPDGMFKTAPYGEAGAQFVDYVTNYNQQTVPVTKQHSDAQGNQWYLATVNGTQYWIDQRSFSPVVTKVVDYQAKIVPRTTRDGVFSGAPYGEVNAKLVNMATAYQNQVVHATGEYTNASGITWSQFALSGQEDKLWIDKRALQA

SEQ ID NO: 18 - protein, 1274 aa, Leuconostoc citreum
  MISC_FEATURE (1)..(1): BRS-B D1
DTQTPVGTTQSQQDLTGQTGQDKPTTKEVIDKKEPVPQVSAQNVGDLSADAKTPKADDKQDTQPTNAQLPDQGNKQTNSNSDKGVKESTTAPVKTTDVPSKSVAPETNTSINGGQYVEKDGQFVYIDQSGKQVSGLQNIEGHTQYFDPKTGYQTKGELKNIDDNAYYFDKNSGNGRTFTKISNGSYSEKDGMWQYVDSHDKQPVKGLYDVEGNLQYFDLSTGNQAKHQIRSVDGVTYYFDADSGNATAFKAVTNGRYAEQTTKDKDGNETSYWAYLDNQGNAIKGLNDVNGEIQYFDEHTGEQLKGHTATVDGTTYYFEGNKGNLVSVVNTAPTGQYKINGDNVYYLDNNNEAIKGLYGINGNLNYFDLATGIQLKGQAKNIDGIGYYFDQNNGNGEYRYSLTGPVVKDVYSQHNAVNNLSANNFKNLVDGFLTAETWYRPAQILSHGTDWVASTDKDFRPLITVWWPNKDIQVNYLKLMQQIGILDNSVVFDTNNDQLVLNKGAESAQIGIEKKVSETGNTDWLNELLFAPNGNQPSFIKQQYLWNVDSEYPGGWFQGGYLAYQNSDLTPYANTNPDYRTHNGLEFLLANDVDNSNPVVQAEQLNWLYYLMNFGQITANDSNANFDSMRIDAISFVDPQIAKKAYDLLDKMYGLTDNEAVANQHISIVEAPKGETPITVEKQSALVESNWRDRMKQSLSKNATLDKLDPDPAINSLEKLVADDLVNRSQSSDKDSSTIPNYSIVHAHDKDIQDTVIHIMKIVNNNPNISMSDFTMQQLQNGLKAFYEDQHQSVKKYNQYNIPSAYALLLTNKDTVPRVFYGDMYQDYGDDLDGGQYMATKSIYYNAIEQMMKARLKYVAGGQIMAVTKIKNDGINKDGTNKSGEVLTSVRFGKDIMDAQGQGTAESRNQGIGVIVSNSSGLELKNSDSITLHMGIAHKNQAYRALMLTNDKGIVNYDQDNNAPIAWTNDHGDLIFTNQMINGQSDTAVKGYLNPEVAGYLAVWVPVGANDNQDARTVTTNQKNTDGKVLHTNAALDSKLMYEGFSNFQKMPTRGNQYANVVITKNIDLFKSWGITDFELAPQYRSSDGKDITDRFLDSIVQNGYGLSDRYDLGFKTPTKYGTDQDLRKAIERLHQAGMSVMADFVANQIYGLHADKEVVSAQHVNINGDTKLVVDPRYGTQMTVVNSVGGGDYQAKYGGEYLDTISKLYPGLLLDSNGQKIDLSTKIKEWSAKYLNGSNIPQVGMGYVLKDWNNGQYFHILDKEGQYSLPTQL

SEQ ID NO: 19 - protein, 1774 aa, Leuconostoc fallax
  MISC_FEATURE (1)..(1): BRS-C
MKQQESITRKKLYKAGKSWVVAATLFAATLFAAMGAAGATTVASADVQKDTVVVTADKNTTDKDKEPIKTAGANVVDKGVAQTTDTNTTDKKTIEVGKSVDMSATDKKVTETVKSVDTSATDKKTTEAVKPVDTNATDKKATEAVKPVDTNATDKKTTEAVKPVDTNTTDKKVTEAIKPVNTNADDKTAEPVKTISATKDTVKTIANKQKGATEEQAVITEGHYEAQGDGFVYITKDGKQLTGLQNINGNTQYFDPATGQQLKGDIKAVAGTVYYFDKNSGNARVYQKVADGTYSENNEHWQYISKVDNKPVEGLYNVQGNLQYFDMSTGNQVKNDIRSVDGVTYYFDKDSGNGSAFNALSAGEYVEKKETDAQGNQNSYWTYSGLDGNPVKGLYDINGSLQYFDEKNGAQLKGGTATVNGVTYYFEQDKGNLISVVNSVESGQYKIDNDNVYYIDNQGNTLKGLYAINGQLNYFDMSTGVQLKGASENANGVGYYFDKDKGNGQYQYSLITSTLANAFSKHNAANDYTQSSFTHTVDGFLTADTWYRPTEILKNGTTWVASTSQDLRPMITVWWPNKNVQLNYLKLMQTEGLLDSGQVYDLNSDQALLNQAAQTVQVNIEKRITKAGNSDWLNDLLYNSHGETPSFVKQQAIWNADSEYHGGWFQGGYLAYRNSDLTPYANSSYRHYTGMEFLLANDVDNSNPIVQAEDLNWLYYLMNFGTETGNDPQANFDSIRIDAISFVDKQVAKKAYELLHDMYGLSASDAVANKHVSIVEASADQTPVTTENHDALIESYWRDTMKNSLSKDASIDSSAGSLSAMINDGNVDRANDSTTESSIFPNYTIVHAHDKDIQDAVSNVMKIVNNDPSISLDGFTMEQLEKGLSAFYADQRSAVKQYNQYNIPSAYAVMLTNKDTVPRTFYGDMYQDDGQYMANKSLYYDAIDTMMKARLKYVSGGQTMSVTKINNANSQKSGEVLTSVRFGKGVMDATDAGSAESRTQGIGVVVSNSSGLQLNDNDKIVLHMGAAHKNQEYRALMLTTNDGIKSFNNDEAPINYTDDNGDLIFDGHNIDGQENTAIRGYLNPQVAGYLAVWVPTGAKDDQDARTQPSNEKSTDGKVLHTNAALDSELIYEGFSNFQPMPTTKDEYTNVMIAKNIDLFKSWGITNFELAPQYRSSDGKNINDRFIDSLVQNGYGLSDRYDLGFETPTKYGTDQDLRTAIKTLHQAGMTVMADYVANQIYGLNTSQEVVDAQRVNSDNNAVEVRYGQHLNVVNSIGGGEYQNLYGGKYLEILNKLYPDLLVDENGNKIDIDTKIKQWSAKYLNGSNVTGLGMGYVLKDWSNGQYFNISNTDGKVMLPEQLVKHMPAVEIGTQTNYTAYISSTIRRDGLYNNMPWGVTATGQDGNEIKWERQGSTSDYNHQKVQVNRQYVDKQGVVWNLINFDDKDLWVDSNALVTVNFTSQKPTKHFVQFGMRQGKYDGFYLSAPYKQTESKWVASTRTHQGQLLEVVGQYTTGSGSRKVTWYLVGLDGKQVWVDSRAVGTNFSHKTNINLLINSATRNDGMYLNAPYGQKGYKRETSSRFYNEKLVTVSQQYYDNKGVIWNLITLNGKKLWVDSRAFATVIDKKVNQSLYINSRNDGMYLNAPYRAQGAKRYASTKTYTGQRVQVTLQRKDTHGVTWYLTKVDSKQLWVDSHAFAPTFTRNVSLNVKVNSSKRNDGIYLNAPYGNKKAKRIASTKAYNGKRVKASKEYKDAKGVTWYLVNLNNKQVWIDKRAF

SEQ ID NO: 20 - protein, 1375 aa, Lactobacillus kunkei
  MISC_FEATURE (1)..(1): BRS-D_D1
NGQTWKYVSQNKDIQGLYKDNNDQLRYFNEYDGTQAKGDIVNVNNDNYYFDKDSGQGHKIDSYTGGSYSESKVNNQDGWIYKSSDNNDVKGVATVDGNIQYFDQNTGLQLKGGSAQIGGVDYYFDPNKGNLVGKVDQVVNSNDYSDNKLLDSNKNVVKGLVVNNGQLQFFDTSNGNQAKNKQVIANGITYYFDTNGNGQYLFTNTGKSAVDDFTQRNAANSVNPSDYKNVVDGFFTADTWYRPKQILDNGTTWRNSNSNELRPMITAWWPNKDVQVNYLKLMQNNGLLDKSNSYSIQSDQQTLNQAAQKAQVNIEKKISQTGNTDWLNDLLFKGNGDNPSFVKQQYIWSSDSESPWQGDAWFQGGYLKYGNSVMTPNTNSNYRDSNNLFDFLLANDVDNSNPAVQAEDLNWLYYLTNFGTITANDSNANFDSIRIDAVDFISNDIIQRSYDYLRQKFNLMQSDANADSHISLVEGGVDAGTTSYSNDGLVEAPFRLDAYPLLHKQDGDVFKNLIDEEDSGIDISNHNGETNTNNTIGGITLSGGKPNYSIVHAHDKDVQEKVGQAIIDTTGIKDWTDFTPSQLAQGLETFYNDQRQTVKKYNDYNVPSAYAIMLTNKGTVPRIYYGDMYQDDGQFMQKKSLYYDDIANLMTARKKYVSGGQSMVDNNGILTSVRFGKGANTVSDSGTEDTRNQGIGLIVGSAPKKVLNDGDTVVLHMGAAHKNQKYRALMLTTENGIQNYNSDDNAPVAETDDNGDLVFSNKDINGQANTAIKQVANPEVNGYLAAWVPVGASDDQDSRTAPSTSQNNDGNVLHENDALDSNLIFEGFSNFQPTPTNHDEYANVVIAKNASLFKDWGVTSFEMAPQYRSSQDHTFVDSTIDNGYAFSDRYDLGFGTPTKYGTDEDLRNAIKSLHDNGMQVMADVVYNQLYNLPGQEVVSATRAGVTGNTNALPFGTQLYVVNTIGGGDYQKKYGGAFLNELQEQYPSLFKSQKYKYYYKNYANNGAGPGYLTVNDAERSDIPYNQPITEWSAKYMNGTNILGRGMGYVLKDWNTGDYFKLSGSDSTLPSSLTYKSGWVENPDSTWSYYEKNNIDKLTGSQVINEERVFFDNNGIQVKGGWVKNSNGTYSYYDKNSGNILTGDQLIDGEHFFFDNNGVQVKGKWIKNSDGSKSYYDSHLGKLIKTDKKVSSNARKKKSKEELLYENALKVLRKDKKRLDKNKTKANIRKYNKSLKKYRKAKKKLLAITKNRVANARKAIKIAKKVLSKRKNINNEKRYYKALKEYYVAEKSYLKITGNYNKKYYYEFDKLTPKVKVVKNIYSYKSRHFTKKNRVKKIKKGTLVRVKSIVRSGKVARINIGNGHFITSSKDFIKMFK

SEQ ID NO: 21 - protein, 1232 aa, Leuconostoc mesenteroides
  MISC_FEATURE (1)..(1): BRS-E_D1
QQNNATLQVSPTKNDNSVAKNTTSTANVAKVDITTDNTRDISSANNVNNLITNQYKENSNGSWSYYDNNGQIVKGLQTINGNIQYFDSTTGEQVKGQTLTIDGVIYSFDKDSGNGTKTEVASLPTTGSYATKDGSNWQYEDQQQQPIKGLYTDKGNLRYFNETDGTQVKGTVVSVDNNTYYFDKDSGNGQLVPSVTGGQYGTIQLNNQTVWVYRNANGEIVKGLQNINGNIQYFDPNTGEQLKGKVATVNGVTYYFEASDGNLVGTVSDGLVTVNGQIQYFDPATGEQAKNKQVIVNNVTYYFDDNGYGQYLFTNAILSTTPDAYSAHTQAYNTDQSSFTNVVDGFLTADSWYRPKEVIADATGSAWQTSSENDYRPIITVWWPNKNVEVNYLKLMQDNDLLSTQTQFTIFSDQYTLNEAAQAAQNEIEKRIYREKSTDWLKDLLFEAHGDTPSFVKQQFIWNKDSEYQGAGGGNLWSLQGGYLKYVNDSETSWSDSTSRKHDYYEYLLGNDIDNSNPQVIAENINWLYYLMNFGSLTGNDDTANFDGVRMDAVIYMKGEASTKVYQFLHKSDELTKNEKIANEHISIVEDGTDETTKNNSALIVSKWNENIASSLAKIASGKDTSLEALVKTDEKTSVSRIMNSSVESDVNPNYSMIRSHDRGSQDEVINASKVANNDQSIALDNINLNQLENGLKLYYEDQASPTKNYNYYNIPASYALLLSNKDTVPRLYYSDMYQDYDQNPDTPQQYMSKPTIYYSAIDALLKARIKYVAGGQSMAVEKVGDNKDQEVLTSVRYGKNVMTATDTGTVESRTEGMGVIVSNNTKLKLSTTDQIVLHMGAAHANQAYQALMLTNDEGIQLYNDNAPVVWTDHNGDLVFNGNDINGQKNTSIKGYFNPQVAGYLAVWVPVGATDTQDARTKASTNATTDGKVFHSNAALDSNVIYEGFSNFQPIARNHNDFSNVKIAENVDLFKKWGITSFELAPQYRSADVSDLVGSTFVDVVTKNGYGLSDRYDLGFVTPTKYGSDSDLRNAISSLHAQGIQAMADFVGNQIYALNDGQEVVTAQRSDMFNNTLNNAFGTELYVVNSIGGGKYQAKYGGNYLEEIASLYPDLFTNQDGTKIDINTKIKQWSAKYMNGTNVLGRGMGYVLKDWNTATYFKLDGEHTVLPAALTLSGLKVENGVTYYYKNNERQTGTQTVDDVTYFFDPKTGAMKKDYFDFTADNKVYYYG

SEQ ID NO: 22 - protein, 1672 aa, Fructobacillus tropaeoli
  MISC_FEATURE (1)..(1): BRS-F
MRKKLYKSGKMWVAASVAISFSALSISVGNNGAKADDSQQSSTQIQSTQVTTALPAGGQYSTTNGGQSWNYLVNGVAIKGMYQDGQGQLRYFNFIDGTQVKGEFLSINGTYYYFDQNSGEGHLVPTQSNGHYTEIGNTGAWGYQNSNGELVKGIQNIDGQLRYFDENTGNQVKGGSATIGNKSYYFEPSQGTLTTTIDQVSDAQNANIRGLATVNGQLNYFDPTTGEQAKHKQVATNGATYYFNDSGVGTYLFTNVQNTPANDVSQHNAVNSTDTKDYTNTVDGFLTADTWYRPKYILDNGENWRASNEGEYRPFIMNWWPNKNVEVNYLKLMQNNNLLSSTVQYDLFTDQAILNQAAYQAQIAIEKRIKSEGSTDWLNTLLFGGDDSHPSFVKQQFIWNSDSESPWQGDAWFQGGYLKYGNSVMTPTSNSNYRQAGNAFDFLLANDVDNQNPIVQAEDLNWLYYLMNFGSITTNGLDNDSNFDSIRLDAVDFIHNDAIQRTYDYLRQAFNLTKNEATANQHLSLVEAGVDAGTTTYNSDGLIESNIRPLATDSLTNAPGKNASLSNLIKDVDSGEVIADHANFSTDDGIPNYSIIHAHDKGIQENVGAAITAATGADWTNFTTEQLEQGLDLYYQDQRSTNKKYNIYNLPSIYALMLTNKGTVPRVYYGDMYQDNGQYMQQKSLYYDAISSLMTARKQYVAGGQTMSVDENGLLKSVRFGKNAMTAQDTGDAETRTEGVGVIIGNDPSVKVADGQTVTLDMGAAHKNQAYRPLILTTSDGIQTYDSDENAPVVYTDDNGILTFSNQDINGQANTKIVGTLNPQVSGYLAVWVPVGASADQDARTAPSTQSTNDGKVLHTGAALDSNLIFEGFSNFQPMPTTHDEMTNVVISQNASQFAKWGITSFEMAPQYRSSEDHSFLDSTIDNGYAFSDRYDLGFGTPTKYGTDEDLRNAIKALHQNGMQVMADVVMNQLYSLNGKEVVSASRAGVYGNDVDLPFGTQLYVVNTTGGGEYQKKYGGAFLNIIKEKYPTLFDSESYDYYLKNYSDNGHGPAYMTTATATREAIPSDQPLKEWSAKYMNGTNILGLGMGYVLKDWNNGAYFKLSGTDTTLPQSLVALTGWNQNDDGTWSYYSTDTDDRVTGKQVIDGRTLLFDNQGNQIKGGWGENPDGTWSYYNADTGDRVIGEQVIDGRTLFFDNQGVQVKGGWGENYDGTWSYYNADTGDRVTGKQVIDGRTLLFDNRGVQVKGGWGENSDGTWSYYNADTGDRVTGNQLIGGRNLLFDNQGNQIKGGWDENPDGTWSYYNADTGDRVTGVQVIDGKQLLFDSNGIQVKNSWQKNANGTWSYYDANDGHLVPANSSNDGTSSSTQDSGNKSNQNPSSSSNAVNKTTGWIKNSDGTWSYLSAKSGQKVTGSQTIDGKQLLFDDHGVQIKGGWGKNADGTWSYYDANSGELTSTSDMSNVNPQQTTTTTNEQSTTNQPTDITKNSDGVYVYKNDSNKKAQGYLNDGSSWKWFNDGQLYTGFQNYMGAYYYFINGIRQQNQWENIWGLKYYVGDDGRTVEGIHAIDGHAYDFGTDGTFNVKGSASGYLNDGKSWMWYEGGNPYTGFRYYMDTYYWFENGVRQDNAWHQAWGLTYYTGADGRAVQGVQNINGKLYYFGNDGTFFMRTNQEV

SEQ ID NO: 23 - protein, 1693 aa, Lactobacillus kunkeei
  MISC_FEATURE (1)..(1): DSR-G CD2
YGLKLIDGKMKYVSTGGFIAKNTFIQDDNHNWYYVDNNGDFVTSPQVINGNKYFFLSNGVNLRDFISVNSDGTMNYYQSNGILANNPGYYYSNNINQMVHVNNNGVLDTGIVNVNGYVQYFDDNGYQVKGDIVNFNGRKMYFDDGSGNLVYNRFVSYNGNWYYAGSNGLLVNGLQNINNQSLYFDDNGKQVKGGMISLDNHKMYFDANSGNLYKNSFVLLDGDVYYANNDGYIVNGYQNIGGRNLYFDSDGKQVKDQFVNINGNKVYFNGTDGSEVKDDFIIHDDKEYYADNQGHLLTGYNFVNGQNMYFNEDGSQVKNSIVSLDGKLMYFDENSGNQVKNGFILHNGNVYYSNKDGILVTGYQNIDGQDLFFNADGTQIKGGTAEIDGVNYYFENGEGHLVGKVDQVINSNRFSDNKLLDANNNVVKGLIFNNGQLQYFDSLTGDQAKNKQIIADGNTYYFDNSGNGTYLFTNIGESETNDFSQRNAANSVNLSDYKNVVDGFFTADTWYRPKQILDNGTTWRNSNSNDFRPMITVWWPNKNVQVNYLKLMQNNNLLDKSTNYTLQSNQQMLNQAAQNAQVNIEKKIAQTGNTDWLNDLLFKGNGDTPSFVKQQYIWNILSESPGQDDALLQNGYFKYVNSELTPNTNSSYRISNNLCDFLLANDVDNSNPAVQAEDLNWLYYLTNFGTITANDPNANFDSIRIDAYAFINNDIVQRSNDYMKQKYKLTESSNNANSHLSIVEAGVDAGTTSTNNDELVESPFRTISYGLIHKDRNPQDMNNFIKEVDTGVVIADHENNSQEIGQPNYSIVHAHDKDIQDKVGEAIVDATGIKDWTNFTPSELSAGLKLYYDDQRSSEKKYNDYNIPSAYAIMLTNKGTVPRIYYGDMYQDDGQFMQKQSVYYDDIVSLLTARKKYVSGGQSMSVDHNNFLESVRFGKGAGSESDSGNAETRNEGIGLIVGNDQNKKLNDGDTVVLHMGAAHRNQKYRALMLTTNDGIKNYDSDENAPIAETDSNGDLVFSNKDINGQANTSVRGVLNPEVAGYVAAWVPLGASDDQDSRTLSSNKSYNDGKVLHSGDDLDSNVIFEAFSNFQPEPTNENEYENVVIPQKASLFKDWGITSFELPPQYRSSNDHTFVDATINNGYAFSDRYDLGFGQPTKYGTDVDLRNTIKSLHDNGMQVMADVVYNQLYNLPGQEVVSAVRAGFTGNTVSLPFGNQLYVVNTVGGGDYQKKYGGAFLQKLYQEYPSLFDSEKYQYNSKNYVTDLLVMTDGERSAIPSDQPITEWSAKYMNGTNILGRGMGYVLKDWNTGTYFKLNGNNSVLPDVLTYRNSFDDNTGTQSSSSAQSSASNHETQISSSAQSSASNHEAQSSSSAQSSVSSHEVQSSSSAQSSASSHEVQSSSSAQSSASSHEVQSSSSAQSSASSNEAQSSSSAQSSASSHEAQKSSSAQSSASSHGEQGKTSAHGNGNGVSQNKPSQSHANDDSDKVLSNRKLTTNNQVSNDHSVNNQTTQHVSNVNTLSRTKTRENQKIQSKMLTTKSLKQVNKQASSQEKLVKKELKQLDKLKAELKKHSSKKLKKEYNALLQKYKANKKYYLALVAEQNTVEKYLKSAKNLKKDKKALNSLNKKLAKDKKELKRHTSKKLKKTYEKDIKGKKKLTKAIKKYKGDEAKLKKAIVNNFKKASSMKSKKK